(19) United States
(12) Patent Application Publication
(10) Pub. No.: US 2016/0160270 A1
(43) Pub. Date: Jun. 9, 2016

(54) METHODS FOR MODELING CHINESE HAMSTER OVARY (CHO) CELL METABOLISM

(71) Applicants: Markus J. HERRGARD, Kgs Lyngby (DK); Nathan E. LEWIS, San Diego, CA (US); Anders Bech BRUNTSE, Kgs Lyngby (DK); TECHNICAL UNIVERSITY OF DENMARK, Kgs Lyngby (DK)

(72) Inventors: Markus J. Herrgard, Kgs Lyngby (DK); Lasse E. Pedersen, Kgs Lyngby (DK); Nathan E. Lewis, San Diego, CA (US); Anders Bech Bruntse, Kgs. Lyngby (DK)

(21) Appl. No.: 14/905,781

(22) PCT Filed: Jul. 18, 2014
(86) PCT No.: PCT/US14/47296

§ 371 Date: Jan. 15, 2016

Related U.S. Application Data
(60) Provisional application No. 61/856,526, filed on Jul. 19, 2013.

(51) Int. Cl. C12Q 1/68; C40B 30/02; G06F 19/22

(52) U.S. Cl. CPC C12Q 1/6827; G06F 19/22; C40B 30/02

(57) ABSTRACT

Embodiments of the present invention generally relate to the computational analysis and characterization biological networks at the cellular level in Chinese Hamster Ovary (CHO) cells. Based on computational methods utilizing a hamster reference genome, the invention provides methods for identifying a CHO cell line having a desired genetic trait, as well as for generating a desired CHO cell line having a genetic basis for a desired phenotype. Additionally, described herein are methods for constructing and analyzing in silico models of biological networks for CHO cells.

### CROSS REFERENCE TO RELATED APPLICATION(S)

This application claims the benefit of priority under 35 U.S.C. §119(e) of U.S. Ser. No. 61/856,526, filed Jul. 19, 2013, the entire content of which is incorporated herein by reference.

### BACKGROUND OF THE INVENTION

1. Field of the Invention

The present invention relates generally to systems biology and more specifically to the use of genomic and computational analysis for bioproduction of biological molecules.

2. Background Information

Recombinant therapeutic proteins are increasingly important to the pharmaceutical industry. Global spending on biologics, such as antibodies, hormones and blood factors, reached $138 billion dollars in 2010. Chinese hamster ovary (CHO) cell lines. CHO cells are a cell line derived from the ovary of the Chinese hamster, often used in biological and medical research and commercially in the production of therapeutic proteins. They were introduced in the 1950s, are grown as a cultured monolayer and may require the amino acid proline in their culture medium. CHO cells are used in studies of genetics, toxicity screening, nutrition and gene expression, particularly to express recombinant proteins. Today, CHO cells are the preferred host expression system for many therapeutic proteins, and the cells have been repeatedly approved by regulatory agencies. Moreover, they can be easily cultured in suspension and can produce high titers of human-compatible therapeutic proteins.

To date, most improvements in CHO-based recombinant protein titer and quality have been achieved by random cell line mutagenesis and media optimization. Meanwhile, efforts to engineer mouse cells have greatly benefited from numerous genomic tools and technologies, owing in large part to the availability of the Mus musculus reference genome sequence. Genomic resources are also now being made available for CHO cells, such as the CHO-K1 genome, EST and BAC libraries and compendia of proteomic and transcriptomic data. However, much like how murine cell line data are routinely studied in the context of the Mus musculus reference genome, there is a need for a standard reference for all CHO cell lines to contextualize all of these valuable genomic resources.

Many recombinant protein-producing CHO cell lines were derived from the CHO-K1, CHO-S and DG44 lineages. Each has undergone extensive mutagenesis and clonal selection. Hence, a standard reference genome that is representative of the genomic sequence of all native CHO genes and regulatory elements would be advantageous for the successful implementation of genomic resources in CHO-based bioprocessing.

Glycosylation serves essential functions on many proteins produced in biopharmaceutical manufacturing, making it mandatory to thoroughly consider its biogenesis during the production process. Glycoengineering efforts involve the rational design of glycosylation through adjustments in culturing conditions or genetic modifications. Computational models have been developed to aid this process, aiming to offer cheaper and faster alternatives to try-and-error screening strategies. Such models have included statistical models that correlate environmental factors, nutrients, and/or knowledge of the recombinant protein of interest with glycoprofiles. In addition to these, mechanistic models of glycosylation have been used to predict glycoprofiles. However, these approaches and mechanistic models of metabolism could be integrated to successfully predict glycosylation on products of industrial relevance. Furthermore, systems-level analyses of glycan diversity could elucidate deeper insights into the mechanisms underlying glycosylation, thus feeding back into the development of refined models and enhanced predictive power. While large scale integrated models of metabolism and glycosylation have not done previous to this work, such models could made be organism-specific if based on the genomic content of the organism of interest, and then further tailored to account for the unique glycosylation capabilities of cell lines using transcriptomic data, proteomic data, or mutations.

Organisms in all domains of life rely upon glycosylation and other post-translational modifications for diverse biochemical and physiological functions, such as modulating protein stability, mediating protein-protein interactions in cell adhesion or signaling, facilitating cell-cell communication, or evading recognition by other organisms. Indeed, proper glycosylation is often critical to the development and survival of an organism. These post-translational modifications often involve the covalent addition of glycans to either the amino group of an asparagine (N-linked) or the hydroxy group of a serine or threonine (O-linked). In eukaryotes, glycosylation predominantly occurs in the endoplasmic reticulum and Golgi apparatus, where membrane-bound glycosyltransferases and glycosidases sequentially add or remove monosaccharides, thereby creating a growing sugar side chain of variable length and diverse types of branching (called antennarity), leading to a vast diversity among glycans. Specific glycosylation sites on a protein may or may not carry a glycan (referred to as macroheterogeneity) and different copies of the same protein may carry different glycans on the same site (referred to as microheterogeneity). While this glycan heterogeneity may suggest that many functions of glycosylation do not require specific glycans, glycosylation is clearly not a purely random process. In fact, at certain sites, glycoproteins are highly sensitive to their glycosylation, as subtle alterations in glycan structure can result in protein malfunction, possibly implicating fatal consequences, such as failed development, disease, or cancer. However, glycosylation occurs without a template, in contrast to protein or nucleic acid synthesis, and the molecular mechanisms by which the cell achieves non-random glycan assembly remains poorly understood.

The physiological importance of glycans and our incomplete understanding of their synthesis present major challenges of controlling glycosylation in biotechnological protein production. Glycosylation can critically affect efficacy, serum half-life, or antigenicity in recombinant proteins, and variations in culturing conditions, host glycosyltransferase expression levels, and genetic mutations can potentially influence the glycosylation pattern. Screening of cell lines and culture conditions has facilitated the implementation of the “Quality by Design” principle in biopharmaceutical manufacturing, but rational glycoengineering would be greatly facilitated with predictive computational tools. Such models ideally could be leveraged to predict optimal culturing conditions and production cell line genome engineering efforts to produce desired glycosylation patterns, thus reducing the need for costly screening. Such efforts are now being enabled by recent experimental, analytical, and computational advances in systems glycobiology including novel modeling tools.

Many factors in a cell and its environment influence glycosylation and their contributions can been explored using computational models. In addition, component measurements, such as glycosyltransferase expression levels, nucleotide sugar concentrations, and miRNA regulation could now be readily integrated in these models at a systems level. However, it is important to remember that glycosylation depends not only on the cell's physiology, but also on the structure of the individual protein to be glycosylated. The amount of glycan processing that occurs will be influenced by the enzymatic accessibility to a particular glycosylation site as well as the protein's retention time in certain Golgi compartments. In this respect one would, for instance, expect to find largely unprocessed high-mannose glycans when protein structure limits accessibility to glycosidases and glycosyltransferases, for example by blocking sites by folding or exhibiting incompatible surface charge around the particular site. Currently, our understanding of these processes is limited. Future studies may be able to integrate these factors into computational models, but so far kinetic parameters for each reaction in the network models require calibration to match experimental data before using the model for in silico predictions and each protein class may require its own calibration. Since industrial biotechnology currently focuses on a relatively small number of protein products, efforts to calibrate model parameters for individual proteins may be valuable. Such efforts would also allow the use of smaller glycosylation models that are specific to the glycans synthesized on the recombinant protein of interest. For example, while modeling O-linked glycosylation, one study presented a framework that sampled smaller models derived from all possible reactions and the measured glycans. Using a set of smaller models, researchers have identified putative pathways that synthesize the sialyl Lewis-X (sLe^{X}) epitope on O-glycans relevant to leukocyte cell adhesion, and predicted sialyltransferases whose expression correlates or anticorrelates with sLe^{X} abundance.

In order to circumvent parameter calibration and to enable more global discoveries on the mechanisms driving glycosylation, it would be desirable to develop models that predict glycosylation de novo from protein sequence data. One study applied neural network modeling to link the glycan branching type (high-mannose versus complex) to three possible predictors: primary amino acid sequence, secondary structure and site accessibility. After training the model on a CHO protein dataset using cross-validation, a combination of secondary structure and site accessibility proved to yield good predictions of the glycan type. This result is further supported by a recent meta-analysis showing significant correlation between glycan complexity and site accessibility. Thus, with adequate structural data, these approaches could be a first step towards a general glycosylation model that incorporates recombinant protein sequence, abundances of the protein secretion pathway components, and spatial information to predict macro- and microheterogeneity for an arbitrary protein.

Complex de novo models and calibrated kinetic models offer valuable predictive frameworks to describe the dynamics of glycan synthesis. However, the complexity of these frameworks poses challenges in model development and analysis. For example, parameters for kinetic models are often collected from different publications that studied enzyme kinetics under different conditions and sometimes different species. Similarly, de novo approaches will require protein structure prediction and computationally intensive structural analysis algorithms. In an alternative approach, one study developed a parameter-free representation of the glycosylation reaction network and demonstrated the possibility to gain qualitative insights into the modularity of glycosylation and the impact of transcriptional regulation on glycans synthesis while avoiding the challenge of reconciling model parameters. Constraint-based modeling is another framework that uses few parameters, and could be particularly valuable for integrating glycomics with whole-cell metabolic models, especially given the wealth of well-developed modeling tools available for this framework. Thus, for certain types of predictions, parameter-free and constraint-based approaches will be of particular value.

Mathematical frameworks for modeling of glycosylation are now achieving the complexity that mirrors glycan diversity on a realistic scale, and these models are showing increasing promise to aid in recombinant protein production. To facilitate the interpretation of these complex models, helpful visualization tools have been developed in recent years. Most current models account for enzyme kinetics, but due to the complexity of factors influencing protein secretion, models require parameter calibration to enhance predictive accuracy. These calibration experiments may be circumvented as parameter-free approaches are developed or as models better account for individual protein structure. Furthermore, small-scale models may be especially helpful in applications where glycoform complexity has been successfully reduced to meet specific biotechnological needs. Irrespective of the modeling framework, it is clear that systems glycobiology holds a great potential to augment experimental advances in recombinant protein production, and glycobiology in general.

It is apparent that there exists a need for a model that describes the CHO cell reaction network, in particular the CHO cell metabolic network, which can be used to simulate many different aspects of cellular behavior under different conditions. Additionally, it is apparent that there exists a need for advanced glycosylation models of CHO cell-lines to ensure reliable recombinant protein production having specific glycosylation patterns, in particular the production of antibodies and antibody fragments, multispecific antibodies, fragments and single-chain constructs, peptides, enzymes, growth factors, hormones, interleukins, interferons, glycans, and vaccines. The glycosylation models need to be integrated with genome-scale models of cellular metabolism in order to accurately predict effects of changes in precursor supply and cultivation conditions on glycosylation patterns. Furthermore, these integrated models need to be associated with the genomic sequence and annotation in order to predict how genetic variants unique to different CHO clones to changes in metabolism and glycosylation.

The present invention satisfies these needs, and provides related advantages as well. The wealth of detailed biochemical information available for mammalian cells, including CHO cells, combined with the sequencing and annotation of the hamster and CHO genome sequences, has enabled the first comprehensive, bottom-up reconstruction of the global CHO cell metabolic network as well as methods which utilize such information.

Previous efforts also provide a draft genome of the CHO-K1 ancestral cell line which may be utilized as described in Xu et al. (Nature Biotechnology. 29:735-741 (2011)). The assembly comprises 2.45 Gb of genomic sequence, with 24,383 predicted genes. Most of the assembled scaffolds were associated with 21 chromosomes isolated by microfluidics to identify chromosomal locations of genes. Furthermore, genes involved in glycosylation were investigated, which affect therapeutic protein quality, and viral susceptibility genes, which are relevant to cell engineering and regulatory concerns. Homologs of most human glycosylation-associated genes were found to be present in the CHO-K1 genome, although 141 of these homologs are not expressed under exponential growth conditions. Many important viral entry genes were also found to be present in the genome but not expressed.

In addition to glycosylation models, CHO cell line genomes may also be exploited in genome-scale metabolic models. An example of a human genome-scale metabolic model was described in Thiele et al. (Nature Biotechnology. 31:419-425 (2013)) which describes Recon 2, a community-driven, consensus ‘metabolic reconstruction’, which is the most comprehensive representation of human metabolism that is applicable to computational modeling. This reconstruction accounts for the many catabolic and anabolic pathways known in human, and includes the enzymatic and transport activities of proteins encoded by 1798 human genes. Using Recon 2 changes in metabolite biomarkers were predicted for 49 inborn errors of metabolism with 77% accuracy when compared to experimental data. Using protein expression data, the authors automatically generated a compendium of 65 cell type-specific models, providing a basis for manual curation or investigation of cell-specific metabolic properties. Recon 2 is freely available on the world wide web at humanmetabolism.org/. The invention presented here includes the development of a genome-scale model of hamster metabolism based on the hamster genome, which serves as a stable reference genome for further CHO cell work. This model is further coupled to glycosylation, and genomic and transcriptomic data from less stable CHO cell lines is used to build and analyze cell line specific models and assess their metabolic and glycosylation capabilities.

### SUMMARY OF THE INVENTION

The invention relates generally to use of genomic and computational analysis in bioproduction of biological molecules utilizing CHO cell lines.

Accordingly, in one aspect, the invention provides a method for identifying a Chinese Hamster Ovary (CHO) cell line having a desired genetic trait. This includes: providing a sample CHO cell line genome, or portion thereof; comparing the sample CHO cell line genome, or portion thereof, with that of a reference hamster genome to identify at least one of single-nucleotide polymorphisms (SNPs), indels, inversions, and copy number variations (CNVs) in the sample CHO cell line genome, or portion thereof, thereby identifying variations in the sample CHO cell line genome, or portion thereof, associated with the desired genetic trait, wherein the desired genetic trait is related to an improved function relevant to bioprocessing, thereby identifying the CHO cell line as having the desired genetic trait. In embodiments, the comparison includes performing computational analysis using a computer generated algorithm. The computer generated algorithm is operable to align and map sequence data of the sample CHO cell line genome to that of the reference hamster genome. Additionally, the aligned sequence data is fragmented and sorted according to a mapped position. In various embodiments, the desired genetic trait is related to cell growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

In another aspect, the invention provides a method for generating a desired CHO cell line having a genetic basis for a desired phenotype. The method includes: providing a sample CHO cell line genome, or portion thereof; comparing the sample CHO cell line genome, or portion thereof, with that of a reference hamster genome to identify at least one of single-nucleotide polymorphisms (SNPs), indels, inversions, and copy number variations (CNVs) in the sample CHO cell line genome, or portion thereof, thereby identifying variations in the sample CHO cell line genome, or portion thereof, associated with a desired phenotype; and introducing one or more genetic changes into the sample CHO cell line to produce the desired CHO cell line having a genetic basis for the desired phenotype. In embodiments, the comparison includes performing computational analysis using a computer generated algorithm. The computer generated algorithm is operable to align and map sequence data of the sample CHO cell line genome to that of the reference hamster genome. Additionally, the aligned sequence data is fragmented and sorted according to a mapped position. In various embodiments, the desired phenotype is related to an improved function relevant to bioprocessing, such as cell growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

In another aspect, the invention provides a method for predicting a CHO cell physiological function. The method includes: a) providing a data structure associated with a CHO cell physiological function, the data structure relating a plurality of CHO cell reactants to a plurality of CHO cell reactions, wherein each of the CHO cell reactions comprises one or more reactants identified as a substrate of the reaction, one or more reactants identified as a product of the reaction and a stoichiometric coefficient relating the substrate and the product; b) providing a constraint set for the plurality of CHO cell reactions; c) providing an objective function; and d) determining at least one flux distribution that minimizes or maximizes the objective function when the constraint set is applied to the data structure, thereby predicting a CHO cell physiological function related to the gene. In embodiments, the method may further include generating a computational model. In various embodiments, the CHO cell physiological function is cellular growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

### BRIEF DESCRIPTION OF THE DRAWINGS

FIGS. 1a-1b are graphical representations of gene families across C. griseus and several mammalian genomes. FIG. 1a illustrates that the majority of mammalian genes are orthologous, with more than five thousand preserved as single copies in each species. A few thousand have species-specific duplications, whereas other orthologs were only shared by some of the nine mammals studied here. A small fraction of genes were unique to just one species, and occasionally had paralogs in that one species. FIG. 1b illustrates that the overlap of orthologous gene clusters is shown among the CHO-K1, C. griseus, M. musculus and R. norvegicus genomes. ENSEMBL (v58) annotated genes were used for the CHO-K1, M. musculus and R. norvegicus genomes.

FIGS. 2a-2c are graphical representations depicting genome comparisons between mouse, Chinese hamster, and CHO-K1. Conserved sequences among the mouse, CHO-K1 and C. griseus genomes were determined by aligning their scaffolds (larger than 1 Mb) to the mouse genome. FIG. 2a illustrates assignment of C. griseus scaffolds to M. musculus chromosomes. The C. griseus scaffolds with chromosomal assignment (accounting for more than a quarter of the 2.4 Gb of genomic sequence) were compared to mouse chromosomes to assess the scale of chromosomal rearrangement. FIG. 2b illustrates alignment of CHO-K1 and C. griseus genomes. Few large DNA stretches are missing in the hamster, whereas there are more regions to which CHO-K1 scaffolds could not align. FIG. 2c depicts gene annotations. The number of genes was determined for each “Biological Process” GO slim category in both the C. griseus and CHO-K1 genomes.

FIGS. 3a-3f are graphical representations depicting the mutation landscape of CHO cell lines. CHO cell lines have diverged over time due to numerous iterations of mutation, selection, and clonal isolation. FIG. 3a illustrates the family tree of a few cell lines, with the sequenced lines highlighted in blue. FIG. 3b illustrates sequencing read depth (normalized by the average read depth for the cell line, and averaged over 100 bp bins) for the DHFR gene, a selectable marker for some CHO cell lines. The DHFR gene was clearly deleted in the DG44 cell line, as no DG44 reads aligned to this region. FIG. 3c illustrates that no PCR product was obtained for the gene either. Mutations were further analyzed on a genomic-wide scale. FIG. 3d is a phylogenetic reconstruction based on the diversity of SNPs. The distribution of SNPs recapitulate the known historical divergence of these CHO cell lines from inferred ancestral cell lines (gray parent nodes). A phylogenetic reconstruction based on indels yields a qualitatively similar tree. FIG. 3e is a graphical plot of SNP abundance. FIG. 3f is a graphical plot of indels. The abundance of SNPs (FIG. 3e) and indels (FIG. 3f) varied between the hamster chromosomes, as determined using all scaffolds that could be assigned to specific chromosomes (˜26% of the sequence data).

FIGS. 4a-4c are graphical and schematic representations depicting expression changes and copy number variations of key members of the apoptotic pathways. Apoptosis is a complex network of proteins that integrates several external and internal signals to make decisions about programmed cell death. FIG. 4a illustrates that on average, gene expression levels of pro-apoptotic genes are only slightly lower in CHO-K1, in comparison to the Chinese hamster. However, anti-apoptotic gene expression is significantly higher in CHO-K1 (*: P<0.02, Wilcoxon rank-sum test). FIG. 4b illustrates that when assessing expression of individual genes, pro-apoptotic genes (red) tend to more frequently decrease mRNA expression, whereas anti-apoptotic genes (blue) more frequently increase expression. FIG. 4c is a schematic representation illustrating many major pro-apoptotic (red) and anti-apoptotic (blue) proteins in the context of the extrinsic (brown), intrinsic (red), or survival (blue) pathways. Proteins that have copy number variations are plotted in bar graphs with each bar representing a unique cell line as detailed in the legend, and copy numbers are normalized to the copy number in hamster. Thus, a value less than one suggests a loss of a gene copy, whereas a value greater than one suggests duplication. Details on each gene abbreviation are included in Supplementary Table 21.

FIG. 5 is a graphical representation depicting genome size of Chinese hamster and CHO-K1 cell line estimated by k-mer analysis. The x-axis is depth (X); the y-axis represents the frequency at that depth. Without consideration of the sequence error rate, heterozygosity rate and repeat rate of the genome, the 17-mer of distribution should obey the Poisson theoretical distribution. From the actual data, due to the sequence error, the low depth of K-mer frequency will take up a large proportion. At the same time, for some specific genome, the certain heterozygosis rate can cause a sub peak at the position of the half of the main peak, while a certain repeat rate can cause a repeat peak at the position of the integer multiples of the main peak. The blue trace represents the 17-mer distribution for the Chinese hamster and the red trace for CHO-K1. For the Chinese hamster, the genome is estimated to be 2.7 Gb, while using the same amount of data (˜50×), the CHO K1 genome size was estimated to be 2.6 Gb.

FIG. 6 is a pictorial representation illustrating predicted genes supported by different evidences. The Venn diagram shows unique and shared gene number among different annotation methods. Homolog support includes genes annotated by homolog method of CHO-K1 cell line. De novo support includes genes predicted by AUGUSTUS, GlimmerHMM and Genscan. RNA-Seq support includes genes predicted by transcriptome data.

FIGS. 7a-7d are graphical and pictorial representations illustrating that differences in mutations and copy number variations (CNVs) in cell lines may influence the glycoforms of recombinant proteins, and that mutations may differ between cell lines. 256 unique enzymes associated with glycosylation were identified in the C. griseus genome. FIG. 7a is a pictorial representation showing that many of these enzymes have one or more mutations or CNVs in at least one cell line. These variations are associated with many aspects of glycosylation, such as (b) sugar nucleotide synthesis, (c) O-linked glycosylation, and (d) N-linked glycosylation. The combined effect of differential gene expression (two left columns of grey/orange boxes), and differences in sequence and copy number variations between cell lines (two right columns of boxes) can clearly influence the synthesis of 0- and N-glycans that have been detected on native and recombinant IgG glycoforms (nodes). Numbers on the orange boxes refer to the number of cell lines with a SNP or CNV in that gene.

FIG. 8 is a graphical representation illustrating divergence of different TE categories within the genome. The divergence rate was calculated between the identified TE elements in the genome and the consensus sequence in the TE library used (Repbase™ or RepeatModeler™).

FIG. 9 is a graphical representation illustrating the number of hamster proteins showing homology to retroviral proteins with common retroviral protein domains. There are many hamster proteins that are homologous to retroviral gag and kinase proteins. Furthermore, transcripts for many of these were detected with RNA-Seq. However, the low abundance of env proteins is consistent with the observation that endogenous retroviral elements lacking env genes tend to spread throughout genomes much more frequently.

FIG. 10 is a graphical representation illustrating the amount of sequence data associated with the 11 chromosomes of the female Chinese hamster. BACs were used to associate scaffolds with specific chromosomes, and in total 26% of sequenced genome could be associated with a specific chromosome.

FIG. 11 is a graphical representation illustrating the distribution of mouse chromosome with homology to Chinese hamster scaffolds. Scaffolds associated with each hamster chromosome were aligned to the mus musculus genome to assess the extent to which the genomes have diverged. While each hamster chromosome demonstrated considerable rearrangement, similarities were seen between several hamster chromosomes and mouse chromosomes, such as hamster chromosomes 6, 7, 8, 10, and X, which showed considerable homology to mouse chromosomes 2, 11, 6, 15, and X.

FIG. 12 is a graphical representation illustrating the length distribution of Structural Variants (SVs) identified between the hamster genome and the CHO-K1 genome sequence. The distribution of SVs frequency for different lengths. Most of these variations are shorter than 100 bp.

FIG. 13 is a graphical representation illustrating the distribution of genes among the 11 chromosomes in the hamster genome. BACs and optical mapping data were used to associate scaffolds with specific chromosomes, accounting for 26% of the genomic sequence. All genes in these scaffolds were identified and their distribution is shown here. It is noted that BAC coverage of chromosome 9 was considerably low and no gene-containing regions were found in the regions targeted. The distribution of glycosylation enzymes, mirrored that of all genes.

FIG. 14 is a graphical representation depicting the amount of IgG that can be produced in the C. griseus model as a function of growth rate.

FIG. 15 is a graphical representation depicting biomass accumulation for strains 1 through 6 during a 168 h fermentation based on the C. griseus model.

FIG. 16 is a graphical representation depicting strains 1 through 6's accumulated IgG production indexed to an initial biomass inoculation of 1 g dw based on the C. griseus model. It is clear that neither the very fast growing strain 6 nor the very slow growing strain 1 is optimal for a 168 h fermentation, whereas strain 4, with an intermediate growth rate, is optimal.

FIG. 17 is a graphical representation depicting the cumulative amount of IgG that can be produced in the model as a function of growth rate in the CHO-K1 specific model.

FIG. 18 is a graphical representation depicting the accumulated biomass formed as strains 1 through 6 grow during a 168 h fermentation using the CHO-K1 model.

FIG. 19 is a graphical representation depicting strains 1 through 6's accumulated IgG production indexed to an initial biomass inoculation of 1 g dw, using the CHO-K1 model. It is clear that neither the fast growing strain 6 nor the slow growing strain 1 is optimal for a 168 h fermentation, whereas strain 2 is optimal.

FIG. 20 is a graphical representation depicting the amount of IgG that can be produced in the model as a function of growth rate in the CHO-S specific model.

FIG. 21 is a graphical representation depicting the accumulated biomass formed as strains 1 through 6 grow during a 168 h fermentation based on the CHO-S model.

FIG. 22 is a graphical representation depicting accumulated IgG production for strains 1 through 6 indexed to an initial biomass inoculation of 1 g dw, based on the CHO-S model. It is clear that neither the fastest growing strain 6 nor the slowest growing strain 1 is optimal for a 168 h fermentation, whereas strain 2 is optimal.

FIG. 23 is a graphical representation depicting the glycans that can be produced (x-axis) for each glycosyltransferases that was removed in the model (y-axis). For each glycosyltransferase knockout, glycans that can be produced are shown in black, and the number of remaining glycans is shown. Each glycosyltransferase class is associated with specific hamster genes.

FIG. 24 is a graphical representation depicting removal of enzymatic reactions and transporters from the model, and the ability to synthesize 20 experimentally measured glycans.

FIG. 25 is a graphical representation depicting removal of each gene from the model, and the ability to synthesize 20 experimentally measured glycans after each single gene deletion.

FIG. 26 is a graphical representation depicting removal of enzymatic reactions and transporters from the model, and the ability to synthesize 20 experimentally measured glycans, after also altering the uptake of several metabolites, mimicking a media change.

FIGS. 27a-27b are graphical representations depicting a comparison of glycan synthesis rates for 20 experimentally-measured glycan following changes in media formulations.

FIG. 28 is a graphical representation depicting differences in glycan synthesis rates after changing the same media component in the CHO-S and CHO-K1 specific models.

FIGS. 29a-29b are graphical representations depicting the identification of metabolic pathways that are most affected by SNPs detected in CHO-K1 and CHO-S. SNPs were identified by aligning sequencing data from CHO-K1 and CHO-S to the C. griseus genome. SNPs in metabolic enzymes were analyzed using the Provean software, which scores each SNP as how likely it will be deleterious to protein function, based on sequence conservation. Once deleterious SNPs were identified in CHO-K1 and CHO-S, they were inputted into the cell line specific models presented in Example 5, and their effects on all other metabolic pathways were assessed using flux variability analysis. Metabolic reactions showing >5% decrease in possible metabolic flux were identified, and a hypergeometric test was done to identify metabolic subsystems that were enriched in reactions with a decreased metabolic flux. This process was done for SNPs in (a) CHO-K1 and (b) CHO-S using the CHO-K1 and CHO-S metabolic models, respectively.

### DETAILED DESCRIPTION OF THE INVENTION

The present invention is described partly in terms of functional components and various processing steps. Such functional components and processing steps may be realized by any number of components, operations and techniques configured to perform the specified functions and achieve the various results. For example, the present invention may employ various CHO cell lines, elements, materials, computers, data sources, storage systems and media, information gathering techniques and processes, data processing criteria, computational and statistical analyses, modeling and the like, which may carry out a variety of functions. In addition, although the invention is described generally in the bioproduction context, the present invention may be practiced in conjunction with any number of applications, environments and data analyses; the systems described are merely exemplary applications for the invention.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Described herein is a genome sequence of C. griseus (Chinese hamster; including nucleic acid sequences as set forth in GenBank Accession Nos: AMDS01000000-AMDS01218862, all of which are incorporated herein by reference in their entirety and may be downloaded on the world wide web at ncbi.nlm.nih.gov/nuccore/?term=AMDSO1000000) from which various CHO cell lines have been derived. This reference sequence was utilized to analyze the genomic composition and mutational diversity among multiple CHO cell lines, and to study how sequence variations may affect cellular processes that are of bioprocessing relevance, such as metabolism, apoptosis, and glycosylation. The C. griseus genome will serve as primary reference resources in future analyses of -omics data sets derived from CHO cells, which will also aid in bioprocessing systems analysis and in cell line engineering studies.

Based on computational methods utilizing the C. griseus genomic sequence as a reference genome, the invention provides methods for identifying a CHO cell line having a desired genetic trait, as well as for generating a desired CHO cell line having a genetic basis for a desired phenotype. Additionally, described herein are methods for constructing and analyzing in silico models of biological networks. A computational model can be used to predict different aspects of cellular behavior of CHO cells, thereby providing valuable information for a range of industrial applications. Developing models of biological networks of CHO cells can be used to inform and guide industrial applications utilizing CHO cells, such as bioproduction of protein therapeutics.

Accordingly, in one aspect, the invention provides a method for identifying a Chinese Hamster Ovary (CHO) cell line having a desired genetic trait. The includes: providing a sample CHO cell line genome, or portion thereof; comparing the sample CHO cell line genome, or portion thereof, with that of a reference hamster genome to identify at least one of single-nucleotide polymorphisms (SNPs), indels, and copy number variations (CNVs) in the sample CHO cell line genome, or portion thereof, thereby identifying variations in the sample CHO cell line genome, or portion thereof, associated with the desired genetic trait, wherein the desired genetic trait is related to an improved function relevant to bioprocessing, thereby identifying the CHO cell line as having the desired genetic trait.

In a related aspect, the invention provides a method for generating a desired CHO cell line having a genetic basis for a desired phenotype. The method includes: providing a sample CHO cell line genome, or portion thereof; comparing the sample CHO cell line genome, or portion thereof, with that of a reference hamster genome to identify at least one of single-nucleotide polymorphisms (SNPs), indels, and copy number variations (CNVs) in the sample CHO cell line genome, or portion thereof, thereby identifying variations in the sample CHO cell line genome, or portion thereof, associated with a desired phenotype; and introducing one or more genetic changes into the sample CHO cell line to produce the desired CHO cell line having a genetic basis for the desired phenotype.

The goal of the invention is to exploit particular genetic traits or phenotypes of CHO cell lines in bioproduction. For example, traits or phenotypes which are associated with improved function related to bioprocessing may be advantageously targeted. In various embodiments, improved function relevant to bioprocessing, may include by way of illustration, cell growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

Utilizing the methods provided herein, improved CHO host cells may be generated that exhibit characteristics that enhance bioproduction of biomolecules, such as proteins. Such cell lines may exhibit high level expression of recombinant proteins for reliably increasing recombinant protein production, in particular the production of antibodies and antibody fragments, multispecific antibodies, fragments and single-chain constructs, peptides, enzymes, growth factors, hormones, interleukins, interferons, glycans, and vaccines.

As used herein a “reference hamster genome” encompasses all or a portion of the genome sequence of C. griseus (Chinese hamster; including nucleic acid sequences as set forth in GenBank Accession Nos: AMDS01000000-AMDS01218862, all of which are incorporated herein by reference in their entirety and may be downloaded on the world wide web at ncbi.nlm.nih.gov/nuccore/?term=AMDS01000000). In various embodiments, a portion of the genome sequence may include any fragment of the genome desired, such as an individual gene or portion thereof, multiple genes, viral repeats, indels, one or more SNPs, one or more inversions, one or more CNVs, or one or more scaffolds as determined herein and described by GenBank Accession No.

As used herein “improved function” is intended to mean that a particular cellular function is improved in a CHO cell having a particular genetic basis associated with the improved function phenotype as compared to a corresponding CHO cell which does not have the same or similar genetic basis associated with the phenotype having the improved function.

The methods of the present invention utilize a sample CHO cell line genome. The sample genome may be obtained by a number of methods known in the art. For example, a sample genome may be isolated from a cell of a selected CHO cell line. The genome may be further sequenced and annotated as described herein or by any other method known in the art.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” as used herein encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “polypeptide fragment” as used herein refers to a polypeptide that has an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., Fundamental Immunology (Paul ed., 3d ed. 1993)). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes bivalent or bispecific molecules, diabodies, triabodies, and tetrabodies. Bivalent and bispecific molecules are known in the art.

A “humanized antibody” refers to an antibody that comprises a donor antibody binding specificity, i.e., the CDR regions of a donor antibody, grafted onto human framework sequences. A “humanized antibody” as used herein binds to the same epitope as the donor antibody and typically has at least 25% of the binding affinity. Methods to determine whether the antibody binds to the same epitope are well known in the art, see, e.g., Harlow & Lane, Using Antibodies, A Laboratory Manual, Cold Spring Harbor Laboratory Press, 1999, which discloses techniques to epitope mapping or alternatively, competition experiments, to determine whether an antibody binds to the same epitope as the donor antibody.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain humanized antibody of the invention, e.g., humanized anti-integrin β1 antibody, may bind as a monomer. Other exemplary single chain antibodies may form diabodies, triabodies, and tetrabodies. (See, e.g., Hollinger et al., 1993, supra). Further the humanized antibodies of the invention, e.g., humanized anti-integrin β1 antibody may also form one component of a “reconstituted” antibody or antibody fragment, e.g., a Fab, a Fab′ monomer, a F(ab)′2 dimer, or an whole immunoglobulin molecule.

“Nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). As appreciated by one of skill in the art, the complement of a nucleic acid sequence can readily be determined from the sequence of the other strand. Thus, any particular nucleic acid sequence set forth herein also discloses the complementary strand.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “isolated” or “substantially purified,” when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). The Smith & Waterman alignment with the default parameters are often used when comparing sequences as described herein.

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403410 (1990), respectively. BLAST and BLAST 2.0 are used, typically with the default parameters, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid (protein) sequences, the BLASTP program uses as defaults a wordlength (W) of 3, an expectation (E) of 10, and the BLOSUM62 scoring matrix (see Henikoff& Henikoff (1989) Proc. Natl. Acad. Sci. USA 89:10915)). For the purposes of this invention, the BLAST2.0 algorithm is used with the default parameters.

In one embodiment, the desired genetic trait or phenotype associated with improved function relates to glycosylation. As discussed herein, glycosylation serves essential functions on many proteins produced in biopharmaceutical manufacturing.

As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). The term “trimannose core” used with respect to the N-glycan also refers to the structure Man₃GlcNAc₂ (“Man₃”). The term “pentamannose core” or “Mannose-5 core” or “Man₅” used with respect to the N-glycan refers to the structure Man₅GlcNAc₂. N-glycans differ with respect to the number of branches (antennae) comprising peripheral sugars (e.g., GlcNAc, fucose, and sialic acid) that are attached to the Man₃ core structure. N-glycans are classified according to their branched constituents (e.g., high mannose, complex or hybrid).

The present invention further provides methods for constructing and analyzing in silico models of biological networks of CHO cells. A computational model can be used to predict different aspects of cellular behavior of CHO cells.

The reconstruction of a genome-scale reaction network requires the identification of all its chemical components and the chemical transformations that they participate in. This process primarily relies on annotated genomes and detailed bibliomic assessment as further discussed in the Examples.

In one aspect, the invention provides for in silico characterization of the CHO metabolic map using well-established constraint-based methods that have been applied extensively to microbial cells (Trawick et al. Biochem Pharmacol 71, 1026-35 (2006)). Flux balance analysis and flux variability analysis (Lewis, et al., Nature reviews Microbiology (2012)) were used to analyze the emergent metabolic properties of the CHO metabolic map and to predict cell phenotypes for WT hamster cells, and CHO cell lines based on expression data, media conditions, detected mutations, and to identify genes and reactions that could be perturbed using chemical or genetic means to obtain desired phenotypes. In the metabolic map, perturbation of one reaction leads to perturbations in the others, since they are all connected. Thus, nonintuitive interactions and their phenotypic effects on CHO growth can be predicted based on genetic mutations. This is consistent with examples in human mitochondria that shows that mutations in human enzymes belonging to the same functionally coupled reaction set may have similar phenotypes (Jamshidi and Palsson. Systems Biology of SNPs. Molecular Systems Biology (2006)).

As such, the invention provides a method for predicting a CHO cell physiological function or phenotype. The method includes: a) providing a data structure associated with a CHO cell physiological function, the data structure relating a plurality of CHO cell reactants to a plurality of CHO cell reactions, wherein each of the CHO cell reactions comprises one or more reactants identified as a substrate of the reaction, one or more reactants identified as a product of the reaction and a stoichiometric coefficient relating the substrate and the product; b) providing a constraint set for the plurality of CHO cell reactions; c) providing an objective function; and d) determining at least one flux distribution that minimizes or maximizes the objective function when the constraint set is applied to the data structure, thereby predicting a CHO cell physiological function related to the gene. In embodiments, the method may further include generating a computation model. In various embodiments, the CHO cell physiological function is cellular growth, biological product production, production of a protein, production of an amino acid, production of a purine, production of a pyrimidine, production of an oligonucleotide, production of an glycan, production of a lipid, production of a fatty acid, production of a bioactive small molecule, transport of a metabolite, and glycosylation of a protein or lipid or fatty acid.

The in silico methods described herein, utilize a computational methodology similar to that described in U.S. Pat. No. 8,301,393, which is incorporated herein by reference in its entirety. However, the present invention utilizes data structures specific for CHO cell lines so as to model the reaction networks of such cell lines.

As used herein, the term “data structure” is intended to mean a physical or logical relationship among data elements, designed to support specific data manipulation functions. The term can include, for example, a list of data elements that can be added combined or otherwise manipulated such as a list of representations for reactions from which reactants can be related in a matrix or network. The term can also include a matrix that correlates data elements from two or more lists of information such as a matrix that correlates reactants to reactions. Information included in the term can represent, for example, a substrate or product of a chemical reaction, a chemical reaction relating one or more substrates to one or more products, a constraint placed on a reaction, or a stoichiometric coefficient.

As used herein, the term “constraint” is intended to mean an upper or lower boundary for a reaction. A boundary can specify a minimum or maximum flow of mass, electrons or energy through a reaction. A boundary can further specify directionality of a reaction. A boundary can be a constant value such as zero, infinity, or a numerical value such as an integer and non-integer. Alternatively, a boundary can be a variable boundary value as set forth below.

As used herein, the term “variable,” when used in reference to a constraint is intended to mean capable of assuming any of a set of values in response to being acted upon by a constraint function. The term “function,” when used in the context of a constraint, is intended to be consistent with the meaning of the term as it is understood in the computer and mathematical arts. A function can be binary such that changes correspond to a reaction being off or on.

Alternatively, continuous functions can be used such that changes in boundary values correspond to increases or decreases in activity. Such increases or decreases can also be binned or effectively digitized by a function capable of converting sets of values to discreet integer values. A function included in the term can correlate a boundary value with the presence, absence or amount of a biochemical reaction network participant such as a reactant, reaction, enzyme or gene. A function included in the term can correlate a boundary value with an outcome of at least one reaction in a reaction network that includes the reaction that is constrained by the boundary limit. A function included in the term can also correlate a boundary value with an environmental condition such as time, pH, temperature or redox potential.

As used herein, the term “activity,” when used in reference to a reaction, is intended to mean the amount of product produced by the reaction, the amount of substrate consumed by the reaction or the rate at which a product is produced or a substrate is consumed. The amount of product produced by the reaction, the amount of substrate consumed by the reaction or the rate at which a product is produced or a substrate is consumed can also be referred to as the flux for the reaction.

As used herein, the term “flux distribution” refers to a directional, quantitative list of values corresponding to the set of reactions in a network, representing the mass flow per unit time for each reaction.

As used herein, the term reaction is intended to mean a conversion that consumes a substrate or forms a product that occurs in a biological network. The term can include a conversion that occurs due to the activity of one or more enzymes that are genetically encoded by the CHO genome. The term can also include a conversion that occurs spontaneously in a cell. Conversions included in the term include, for example, changes in chemical composition such as those due to nucleophilic or electrophilic addition, nucleophilic or electrophilic substitution, elimination, isomerization, deamination, phosphorylation, methylation, glycosylation, reduction, oxidation or changes in location such as those that occur due to a transport reaction that moves one or more reactants within the same compartment or from one cellular compartment to another. In the case of a transport reaction, the substrate and product of the reaction can be chemically the same and the substrate and product can be differentiated according to location in a particular cellular compartment. Thus, a reaction that transports a chemically unchanged reactant from a first compartment to a second compartment has as its substrate the reactant in the first compartment and as its product the reactant in the second compartment. It will be understood that when used in reference to an in silico model or data structure, a reaction is intended to be a representation of a chemical conversion that consumes a substrate or produces a product.

As used herein, the term reaction is intended to mean a chemical that is a substrate or a product of a reaction that occurs in a biological network. The term can include substrates or products of reactions performed by one or more enzymes encoded by gene(s), reactions occurring in cells that are performed by one or more non-genetically encoded macromolecule, protein or enzyme, or reactions that occur spontaneously in a cell. Metabolites are understood to be reactants within the meaning of the term. It will be understood that when used in reference to an in silico model or data structure, a reactant is intended to be a representation of a chemical that is a substrate or a product of a reaction that occurs in a cell.

As used herein the term “substrate” is intended to mean a reactant that can be converted to one or more products by a reaction. The term can include, for example, a reactant that is to be chemically changed due to nucleophilic or electrophilic addition, nucleophilic or electrophilic substitution, elimination, isomerization, deamination, phosphorylation, methylation, reduction, oxidation or that is to change location such as by being transported across a membrane or to a different compartment.

As used herein, the term “product” is intended to mean a reactant that results from a reaction with one or more substrates. The term can include, for example, a reactant that has been chemically changed due to nucleophilic or electrophilic addition, nucleophilic or electrophilic substitution, elimination, isomerization, deamination, phosphorylation, methylation, reduction or oxidation or that has changed location such as by being transported across a membrane or to a different compartment.

As used herein, the term “stoichiometric coefficient” is intended to mean a numerical constant correlating the number of one or more reactants and the number of one or more products in a chemical reaction. Typically, the numbers are integers as they denote the number of molecules of each reactant in an elementally balanced chemical equation that describes the corresponding conversion. However, in some cases the numbers can take on non-integer values, for example, when used in a lumped reaction or to reflect empirical data.

As used herein, the term “plurality,” when used in reference to reactions or reactants is intended to mean at least 2 reactions or reactants. The term can include any number of reactions or reactants in the range from 2 to the number of naturally occurring reactants or reactions for a particular cell. Thus, the term can include, for example, at least 10, 20, 30, 50, 100, 150, 200, 300, 400, 500, 600 or more reactions or reactants. The number of reactions or reactants can be expressed as a portion of the total number of naturally occurring reactions for a particular cell such as at least 20%, 30%, 50%, 60%, 75%, 90%, 95% or 98% of the total number of naturally occurring reactions that occur in the particular cell.

As used herein, the term “activate” or activation refers to an effect a compound has on another compound, serving to alter the constraints in a positive manner, such as increasing the activity of a reaction. This includes but is not limited to allosteric and non-allosteric regulation of enzymes.

As used herein, the term “inhibit” or inhibition refers to an effect a compound has on another compound, serving to alter the constraints in a negative manner, such as decreasing the activity of a reaction. This includes but is not limited to allosteric and non-allosteric regulation of enzymes.

As used herein, the term “growth” refers to the production of a weighted sum of metabolites identified as biomass components.

As used herein, the term “energy production” refers to the production of metabolites that store energy in their chemical bonds, particularly high energy phosphate bonds such as ATP and GTP.

The reactants to be used in a reaction network data structure of the invention can be obtained from or stored in a compound database. As used herein, the term “compound database” is intended to mean a computer readable medium containing a plurality of molecules that includes substrates and products of biological reactions. The plurality of molecules can include molecules found in multiple organisms, thereby constituting a universal compound database. Alternatively, the plurality of molecules can be limited to those that occur in a particular organism, thereby constituting an organism-specific compound database. Each reactant in a compound database can be identified according to the chemical species and the cellular compartment in which it is present. Thus, for example, a distinction can be made between glucose in the extracellular compartment versus glucose in the cytosol. Additionally each of the reactants can be specified as a metabolite of a primary or secondary metabolic pathway. Although identification of a reactant as a metabolite of a primary or secondary metabolic pathway does not indicate any chemical distinction between the reactants in a reaction, such a designation can assist in visual representations of large networks of reactions.

As used herein, the term “substructure” is intended to mean a portion of the information in a data structure that is separated from other information in the data structure such that the portion of information can be separately manipulated or analyzed. The term can include portions subdivided according to a biological function including, for example, information relevant to a particular metabolic pathway such as an internal flux pathway, exchange flux pathway, central metabolic pathway, peripheral metabolic pathway, or secondary metabolic pathway. The term can include portions subdivided according to computational or mathematical principles that allow for a particular type of analysis or manipulation of the data structure.

The reactions included in a reaction network data structure can be obtained from a metabolic reaction database that includes the substrates, products, and stoichiometry of a plurality of biological reactions. The reactants in a reaction network data structure can be designated as either substrates or products of a particular reaction, each with a stoichiometric coefficient assigned to it to describe the chemical conversion taking place in the reaction. Each reaction is also described as occurring in either a reversible or irreversible direction. Reversible reactions can either be represented as one reaction that operates in both the forward and reverse direction or be decomposed into two irreversible reactions, one corresponding to the forward reaction and the other corresponding to the backward reaction.

Depending upon the particular environmental conditions being tested and the desired activity, a reaction network data structure can contain smaller numbers of reactions such as at least 200, 150, 100 or 50 reactions. A reaction network data structure having relatively few reactions can provide the advantage of reducing computation time and resources required to perform a simulation. When desired, a reaction network data structure having a particular subset of reactions can be made or used in which reactions that are not relevant to the particular simulation are omitted. Alternatively, larger numbers of reactions can be included in order to increase the accuracy or molecular detail of the methods of the invention or to suit a particular application. Thus, a reaction network data structure can contain at least 300, 350, 400, 450, 500, 550, 600 or more reactions up to the number of reactions that occur in a particular cell or that are desired to simulate the activity of the full set of reactions occurring in the particular CHO cell.

A reaction network data structure or index of reactions used in the data structure such as that available in a metabolic reaction database, as described herein, can be annotated to include information about a particular reaction. A reaction can be annotated to indicate, for example, assignment of the reaction to a protein, macromolecule or enzyme that performs the reaction, assignment of a gene(s) that codes for the protein, macromolecule or enzyme, the Enzyme Commission (EC) number of the particular metabolic reaction, the KEGG pathway identifier of the particular metabolic reaction, or Gene Ontology (GO) number of the particular metabolic reaction, a subset of reactions to which the reaction belongs, citations to references from which information was obtained, or a level of confidence with which a reaction is believed to occur in a particular CHO cell. A computer readable medium of the invention can include a gene database containing annotated reactions. Such information can be obtained during the course of building a metabolic reaction database or model of the invention as described below.

Flux constraints can be placed on the value of any of the fluxes in the metabolic network using a constraint set. These constraints can be representative of a minimum or maximum allowable flux through a given reaction, possibly resulting from a limited amount of an enzyme present. Additionally, the constraints can determine the direction or reversibility of any of the reactions or transport fluxes in the reaction network data structure.

The methods described herein can be implemented on any conventional host computer system, such as those based on Intel® or AMD® microprocessors and running Microsoft Windows® operating systems. Other systems, such as those using the UNIX® or LINUX® operating system are also contemplated. The systems and methods described herein can also be implemented to run on client-server systems and wide-area networks, such as the Internet.

Software to implement a method or model of the invention can be written in any well-known computer language, such as Java, C, C++, Visual Basic, Python, R, PERL, MATLAB, FORTRAN or COBOL and compiled using any well-known compatible compiler. The software of the invention normally runs from instructions stored in a memory on a host computer system. A memory or computer readable medium can be a hard disk, floppy disc, compact disc, DVD, magneto-optical disc, Random Access Memory, Read Only Memory or Flash Memory. The memory or computer readable medium used in the invention can be contained within a single computer or distributed in a network. A network can be any of a number of conventional network systems known in the art such as a local area network (LAN) or a wide area network (WAN). Client-server environments, database servers and networks that can be used in the invention are well known in the art. For example, the database server can run on an operating system such as UNIX, running a relational database management system, a World Wide Web application and a World Wide Web server. Other types of memories and computer readable media are also contemplated to function within the scope of the invention.

A computer system of the invention can further include a user interface capable of receiving a representation of one or more reactions. A user interface of the invention can also be capable of sending at least one command for modifying the data structure, the constraint set or the commands for applying the constraint set to the data representation, or a combination thereof. The interface can be a graphic user interface having graphical means for making selections such as menus or dialog boxes. The interface can be arranged with layered screens accessible by making selections from a main screen. The user interface can provide access to other databases useful in the invention such as a metabolic reaction database or links to other databases having information relevant to the reactions or reactants in the reaction network data structure or to mammalian physiology. Also, the user interface can display a graphical representation of a reaction network or the results of a simulation using a model of the invention.

Once an initial reaction network data structure and set of constraints has been created, a model disclosed herein can be tested by preliminary simulation. During preliminary simulation, gaps in the network or “dead-ends” in which a metabolite can be produced but not consumed or where a metabolite can be consumed but not produced can be identified. Based on the results of preliminary simulations areas of the metabolic reconstruction that require an additional reaction can be identified. The determination of these gaps can be readily calculated through appropriate queries of the reaction network data structure and need not require the use of simulation strategies, however, simulation would be an alternative approach to locating such gaps.

In the preliminary simulation testing and model content refinement stage an existing model may be subjected to a series of functional tests to determine if it can perform basic requirements such as the ability to produce the required biomass constituents and generate predictions concerning the basic physiological characteristics of the particular organism strain being modeled. The more preliminary testing that is conducted, typically, the higher the quality of the model that will be generated. Typically the majority of the simulations used in this stage of development will be single optimizations. A single optimization can be used to calculate a single flux distribution demonstrating how metabolic resources are routed determined from the solution to one optimization problem. An optimization problem can be solved using linear programming as demonstrated in the Examples below. The result can be viewed as a display of a flux distribution on a reaction map. Temporary reactions can be added to the network to determine if they should be included into the model based on modeling/simulation requirements.

Once a model is sufficiently complete with respect to the content of the reaction network data structure according to the criteria set forth above, the model can be used to simulate activity of one or more reactions in a reaction network. The results of a simulation can be displayed in a variety of formats including, for example, a table, graph, reaction network, flux distribution map or a as a modal matrix.

As used herein, the term “physiological function,” when used in reference to CHO cells, is intended to mean an activity of a CHO cell as a whole. An activity included in the term can be the magnitude or rate of a change from an initial state of a CHO cell to a final state of the CHO cell. An activity can be measured qualitatively or quantitatively. An activity included in the term can be, for example, growth, energy production, redox equivalent production, biomass production, development, or consumption of carbon, nitrogen, sulfur, phosphate, hydrogen or oxygen. An activity can also be an output of a particular reaction that is determined or predicted in the context of substantially all of the reactions that affect the particular reaction in a CHO cell or substantially all of the reactions that occur in a CHO cell. Examples of a particular reaction included in the term are production of biomass precursors, production of a protein, production of an amino acid, production of a purine, production of a pyrimidine, production of a glycan, production of an oligonucleotide, production of a lipid, production of a fatty acid, production of a cofactor, production of a hormone, production of a bioactive small molecule, transport of a metabolite, or glycosylation of a protein, fatty acid or lipid. A physiological function can include an emergent property which emerges from the whole but not from the sum of parts where the parts are observed in isolation.

A physiological function of CHO cell line reactions can also be determined using a reaction map to display a flux distribution. A reaction map can be used to view reaction networks at a variety of levels. In the case of a cellular metabolic reaction network, a reaction map can contain the entire reaction complement representing a global perspective. Alternatively, a reaction map can focus on a particular region of metabolism such as a region corresponding to a reaction subsystem described above or even on an individual pathway or reaction.

Methods disclosed herein can be used to determine the activity of a plurality of CHO cell line reactions including, for example, biosynthesis of an amino acid, degradation of an amino acid, biosynthesis of a purine, biosynthesis of a pyrimidine, biosynthesis of a glycan, biosynthesis of on oligonucleotide, biosynthesis of a lipid, metabolism of a fatty acid, biosynthesis of a cofactor, production of a hormone, production of a bioactive small molecule, transport of a metabolite, metabolism of an alternative carbon source, and glycosylation of a protein, fatty acid or lipid.

Methods disclosed herein can be used to determine a phenotype of a CHO cell line mutant. The activity of one or more CHO cell line reactions can be determined using the methods described above, wherein the reaction network data structure lacks one or more gene-associated reactions that occur in a CHO cell. Alternatively, methods can be used to determine the activity of one or more CHO cell line reactions when a reaction that does not naturally occur in a CHO cell line is added to the reaction network data structure. Deletion of a gene or a deleterious mutation (a SNP, an indel, a copy number variation, an inversion, etc.) can also be represented in a model of the invention by constraining the flux through the reaction to zero, thereby allowing the reaction to remain within the data structure. Thus, simulations can be made to predict the effects of adding or removing genes to or from a CHO cell line. The methods can be particularly useful for determining the effects of adding or deleting a gene that encodes for a gene product that performs a reaction in a peripheral metabolic pathway. In one contemplated embodiment, adenovirus vectors are used for in vivo transfer of genes determined in silico to be required for a desired functioning of the metabolic pathway.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Genomic Landscapes of Chinese Hamster Ovary (CHO) Cell Lines as Revealed by the C. griseus Draft Genome

Chinese hamster ovary (CHO) cells, first isolated in 1957, are the preferred production host for many therapeutic proteins. Although genetic heterogeneity among CHO cell lines has been well documented, a systematic, nucleotide-resolution characterization of their genotypic differences has been stymied by the lack of a unifying genomic resource for CHO cells. A 2.4 Gb draft genome sequence is reported herein of a female Chinese hamster, C. griseus, harboring 24,044 genes. Additionally, the genomes of six CHO cell lines from the CHO-K1, DG44 and CHO-S lineages were resequenced and analyzed. This analysis identified hamster genes missing in different CHO cell lines, and detected >3.7 million SNPs, 551,240 indels and 7,063 copy number variations. Many mutations are located in genes with functions relevant to bioprocessing, such as apoptosis, sugar nucleotide biosynthesis and glycosylation. The details of this genetic diversity highlight the value of the hamster genome as the reference upon which CHO cells can be studied and engineered for protein production.

Methods

Sample Preparation and DNA Sequencing

Female Chinese hamsters were obtained. Genomic DNA was isolated from multiple tissues using a modified SDS method. See, Peng et al. Crop Sci. 47, 2418-2429 (2007). Seven different paired end libraries were constructed with 170 bp, 500 bp, 800 bp, 2 kb, 5 kb, 10 kb, and 20 kb insert sizes, using the standard protocol provided by Illumina (San Diego, USA). The sequencing was performed using Illumina HiSeq 2000™ according to the manufacturer's standard protocol. The raw data was filtered to remove low quality reads, reads with adaptor sequences, and duplicated reads prior to de novo genome assembly (See Supplementary Notes below).

Optical Mapping

High molecular weight DNA was obtained from Chinese Hamster tissues. Whole genome shotgun single-molecule restriction maps were generated using the automated Argus system (OpGen Inc., Maryland, USA), based on the optical mapping technology. See, Dong et al. Nature Biotechnology 31, 135-141 (2013); and Schwartz et al. Science 262, 110-114 (1993). Individual DNA molecules were deposited onto silane-derivatized glass surfaces in MapCards™ (OpGen Inc., MD, USA) and digested by BamHI enzyme. DNA was subsequently stained with JOJO fluorescence dye (Invitrogen, CA, USA) and imaged within the Argus system. A total of 28 MapCards™ were processed. The DNA molecules were marked up and restriction fragment size was determined by image processing in parallel with image acquisition. This yielded ˜26× optical data.

Genome Assembly

Similar to the assembly of CHO-K1 genome, SOAPdenovo™ v.1.06 was used to assemble the hamster genome into contigs and scaffolds as well as for gap closure. See, Li et al. Genome research 20, 265-272 (2010). The final genome assembly was 2.4 Gb in length, which is about 89% of the estimated genome. The contig N50 (the shortest length of sequence contributing more than half of assembled sequences) was 26.5 kb and the scaffold N50 was 1.54 Mb (See Table 1 below for statistics on genome assembly). Optical mapping data was used to further assemble the genome into super-scaffolds. The scaffolds were extended according to the optical maps to determine overlapping regions between scaffolds and their relative location and orientation. First, the sequence scaffolds were converted into restriction maps by in silico restriction enzyme digestion by BamHI. These in silico restriction maps were used as seeds to identify single molecule restriction maps of DNA from the corresponding genomic regions by map-to-map alignment. These single molecule maps were then assembled together by using the in silico maps, to produce elongated consensus maps (extended scaffolds). The low coverage regions near the ends of the extended scaffolds were trimmed off to maintain high extension quality. To generate sufficient extension length, the alignment-assembly process was repeated 4-5 times, using the extended scaffolds as seeds for each subsequent iteration. All of the extended scaffolds were then aligned to each other. Any pair-wise alignments above an empirically decided confidence threshold were considered as initial candidates for scaffold connection. Alignments that overlapped substantially with the initial scaffolds were excluded from the candidates. Among the remaining alignments, those with the highest score were considered. The relative location and orientation of each pair of connected scaffolds were used to generate super-scaffolds. This resulted in 6,356 super-scaffolds (>2 kb) with N50 of 2.49 Mb (Table 1).

TABLE 1
Assembly Statistics

| | | | | | Super- | |
|---|---|---|---|---|---|---|
| | Contig | | Scaffold | | scaffolds | |
| | Size (bp) | Number | Size (bp) | Number | Size (bp) | Number |
| N90 | 6,390 | 91,476 | 346,540 | 1,637 | 443,523 | 1,091 |
| N80 | 11,724 | 65,207 | 656,362 | 1,156 | 939,760 | 723 |
| N70 | 16,531 | 48,549 | 950,835 | 853 | 1,417,091 | 519 |
| N60 | 21,461 | 36,190 | 1,249,430 | 634 | 1,994,221 | 378 |
| N50 | 26,761 | 26,456 | 1,544,832 | 461 | 2,491,721 | 271 |
| Longest | 219,443 | — | 8,324,132 | — | 10,797,402 | — |
| (bp) | | | | | | |
| Total size | 2,332,459,831 | — | 2,393,115,851 | — | 2,400,585,184 | — |
| (bp) | | | | | | |
| Total | — | 458,620 | — | 287,210 | — | 286,619 |
| number | | | | | | |
| (≧100 bp) | | | | | | |
| Total | — | 128,107 | — | 6,947 | — | 6,356 |
| number | | | | | | |
| (≧2 kb) | | | | | | |

N## contig (scaffold) size is the length of the smallest contig (scaffold) S in the sorted list of all contigs (scaffolds) where the cumulative length from the largest contig to contig S is at least ##% of the total assembly length.

Chromosomal Assignment of Scaffolds

To assign scaffolds to their respective chromosomes, our optical mapping data were used in conjunction with published BAC end-sequencing and fluorescence in situ hybridization. See, Cao et al. Biotechnology and bioengineering 109, 1357-1367 (2012). Specifically, chromosomal assignments were obtained for each BAC, and then blastn was used to find scaffolds with the highest homology to the BAC end-sequences (E-value<1×10⁻⁵). Scaffolds aligned to BACs from more than one chromosome were filtered from the analysis. Once chromosomal assignments were obtained for scaffolds (Supplementary Table 3, publicly available on the world wide web at nature.com/nbt/journal/v31/n8/full/nbt.2624.html#supplementary-information), they were extended to super-scaffolds based on optical mapping data (Supplementary Table 4, publicly available on the world wide web at nature.com/nbt/journal/v31/n8/full/nbt.2624.html#supplementary-information). From this analysis, it was possible to reliably localize 26% of the genomic sequence to specific hamster chromosomes.

RNA Sequencing and Assembly

RNA was isolated from eight tissues from several Chinese hamsters. Total RNA was extracted using Trizol (Invitrogen, USA). The isolated RNA was then treated by RNAse-Free DNase™. The RNA was subsequently mixed and treated using the Illumina mRNA-Seq Prep Kit™ following the manufacturer's instructions. The insert size of the RNA libraries was about 170 bp, and the sequencing was done using Illumina HiSeq 2000™. Raw reads were filtered out if they contained contamination or were of low quality (more than 10% of the bases with unknown quality). The resulting 5 Gb of RNA-seq data were assembled into transcriptional fragments by Trinity (version: r2011-08-20). See, Grabherr et al. Nature Biotechnology 29, 644-652 (2011). The coverage of the transcripts in the genome assembly was then assessed by mapping the assembled transcriptional fragments to the genome assembly using BLAT. See, Kent. Genome research 12, 656-664 (2002).

Gene Annotation

Gene models were predicted using de novo, homology-based, and transcriptome-aided prediction approaches. For de novo gene prediction, a repeat-masked genome assembly was used. AUGUSTUS™ (Version 2.03) (Stanke et al. Nucleic Acids Res 33, W465-467 (2005)), GlimmerHMM™ (Version 3.02), and Genscan™ (Version 1.0) were utilized for de novo gene annotation. For homology-based prediction, the protein sequences were mapped from the CHO-K1 cell line using BLAT™, with an E-value cutoff of 10⁻², followed by Genewise™ (Version 2.2.0) for gene annotation. Genes with less than 70% identity and 80% coverage in the BLAT™ alignment were filtered. Transcriptome aided annotation was done by mapping all RNA-seq reads back to the reference genome using Tophat™ (Version 1.3.3) (Birney et al. Genome research 14, 988-995 (2004)), implemented with bowtie (Version 0.12.5) (Langmead et al. Genome Biol 10, R25 (2009)). The transcripts were assembled using Cufflinks™ (Version 1.2.1) (Trapnell et al. Nature Biotechnology 28, 511-515 (2010)). Taken together with the assembled transcripts from Cufflinks™, the genomic regions covered by the transcriptome were identified. De novo genes with less than 50% coverage in the transcriptome data were filtered. Finally, the non-redundant gene sets were merged with the homology-based method genes and de novo genes, while filtering transposable element genes identified in the functional annotation. Gene functions were assigned according to the best match of the alignments using blastp (E-value≦10⁻⁵) against the Swiss-Prot™ and UniProt™ databases (Release 15.10). The motifs and domains of genes were determined by InterProScan™ (Version 4.5) (Quevillon et al. Nucleic Acids Res 33, W116-120 (2005)) against protein databases. Gene Ontology IDs for each gene were obtained from the corresponding InterPro™ entry. All genes were aligned against KEGG™ (Release 48.2) proteins, and the pathway in which the gene might be involved was derived from the matching genes in KEGG™. If the best hit of a gene was “function unknown,” “putative,” etc., the second best hit was used to assign function until there were no more hits meeting the alignment criteria (then this gene would be annotated as functionally unknown).

Genome Comparison

The assembled Chinese hamster and CHO-K1 scaffolds (>1 kb) were masked by RepeatMasker™ to remove repeat elements. The repeat-masked mouse genome was downloaded from ENSEMBL (release 60). See, Waterston et al. Nature 420, 520-562 (2002). The repeat-masked hamster and the CHO-K1 assemblies were aligned to the mouse genome as previously described. See, Jax et al. Nature 479, 529-533 (2011). The LASTZ pair-wise whole genome alignment software (on the world wide web at bx.psu.edu/miller_lab/dist/README.lastz-1.02.00/README.lastz-1.02.00a.html) was used with the parameters: K=4500 L=3000 Y=15000 E=150 H=0 O=600 T=2. The Chain/Net package (Kent et al. Proc Natl Acad Sci USA 100, 11484-11489 (2003)) was subsequently used to process the alignment. Structural variations between the hamster and CHO-K1 genomes were found using a procedure previously applied to compare two human genomes. See, Li et al. Nature Biotechnology 29, 723-730 (2011). Large masked scaffolds (larger than 1 megabase in length) were processed with LASTZ using the aforementioned parameter set. These alignments between the hamster and CHO-K1 were corrected for inaccurately predicted gaps in the assembly and other alignment errors. Using the corrected alignments, the best match for each location on the CHO-K1 scaffolds was chosen by the option “axtBest.” This deploys a dynamic programming algorithm using the same substitution matrix as used during the alignment. The hits that contributed most to the colinearity between the large scaffolds of the Chinese hamster and CHO-K1 were selected, and discrepancies between the aligned sections were called as insertions and deletions.

Detection of Sequence Variation Among Cell Lines

Six different CHO cell lines were sequenced to assess the extent of genomic divergence from the hamster genome. The cell lines were grown on their respective media (Supplementary Table 13, publicly available on the world wide web at nature.com/nbt/journal/v31/n8/full/nbt.2624.html#supplementary-information), after which their DNA was harvested and sequenced to greater than the minimum recommended depth of 9× for each cell line, to assure that enough coverage was obtained to resolve heterozygous SNPs. Sequencing data can be obtained from the NCBI short read archive (see Supplementary Table 25 for accession numbers; publicly available on the world wide web at nature.com/nbt/journal/v31/n8/full/nbt.2624.html#supplementary-information).

Missing genes in the six resequenced cell lines and the previously sequenced CHO-K1 ATCC genome were detected as follows. See, Xu et al. Nature Biotechnology 29, 735-741 (2011). Sequencing reads from the seven cell lines and hamster were mapped to hamster assembly with BWA™ (version 0.5.9). Read depth of genes was calculated using ‘depth’ tool of SAMtools™ (version 0.1.18). A gene was declared to be deleted if it conforms to the following criteria. First, when mapping the hamster reads to the assembled hamster genome scaffolds, the read depth of the gene must be greater than half of the mean read depth across all hamster genes. Second, the read depth of the gene for a given cell line must be less than 0.1. SOAPaligner™ (version 2.21) was also used for a repeat trial. The resulting read depth distribution was consistent with that derived from BWA™.

To detect SNPs, indels, and CNVs, the raw reads from each cell line were mapped to the hamster genome assembly to determine sequence variations. To aid the process of variant detection, the hamster scaffolds were concatenated in a random fashion to obtain 12 pseudo chromosomes. SOAP™ was used to align the sequencing reads from each cell line to the reference hamster assembly. The alignments were subsequently split into pseudo chromosomes and sorted according to the mapped position. SOAPsnp™ was used to identify SNPs in each cell line. To further refine the predicted SNPs, an alternative approach was adopted using BWA to align the reads to the hamster assembly. The ‘mpileup’ tool of SAMtools™ was applied to get the information of each genomic position in the different samples and BCFtools™ in the same package was used for variant calling. The two SNP datasets were subsequently combined to make the final SNP dataset. For each library, SNPs with depth less than half of the mean depth were filtered. Also SNPs that were located within 5 bp of another SNP were filtered. In total, 3,715,639 SNPs were identified. SNPs were used to reconstruct the phylogeny of the CHO cell lines. The Jukes-Cantor pairwise distance was computed between all strains and the phylogenetic tree was built using the unweighted pair group method average. The alignments were further processed using SOAPindel™ (on the world wide web at soap.genomics.org.cn/soapindel.html) to identify indels and analyzed using CNVnator to detect CNVs. See, Abyzov et al. Genome research 21, 974-984 (2011).

Accession Numbers

This Whole Genome Shotgun project has been deposited at DDBJ/EMBL/GenBank under the accession No. AMDS00000000. The version described in this study is accession No. AMDS01000000. Accession numbers for the sequencing data for the cell lines and the hamster transcriptome are listed in Supplementary Table 25, publicly available on the world wide web at nature.com/nbt/journal/v31/n8/full/nbt.2624.html#supplementary-information.

Results

Genome Assembly

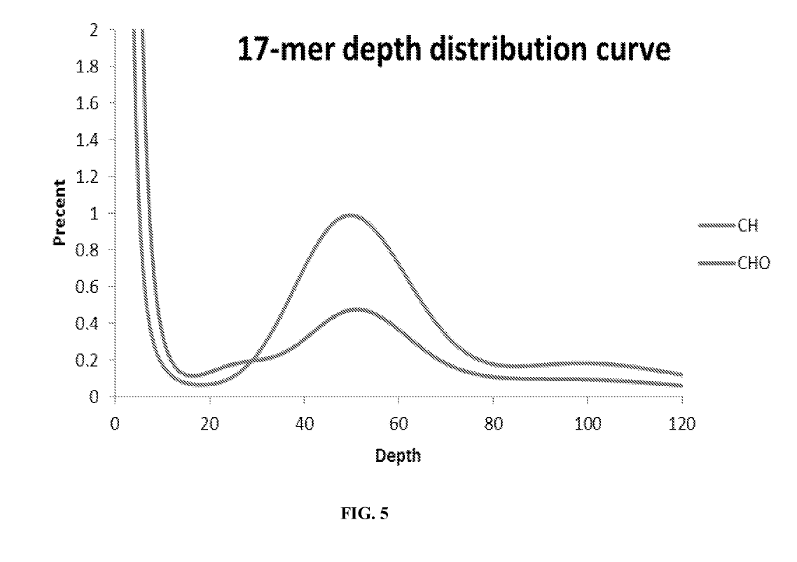

Female Chinese hamster DNA was acquired from various tissues and sequenced using the Illumina HiSeq 2000™ platform, yielding 347.5 Gb of raw data (Supplementary Tables 1 and 2, publicly available on the world wide web at nature.com/nbt/journal/v31/n8/full/nbt.2624.html#supplementary-information). Using SOAPdenovo, 2.4 Gb of the genome was assembled with a contig N50 of 26.5 kb and scaffold N50 of 1.54 Mb (Table 1 above). The genome was further assembled into super-scaffolds with optical mapping yielding an N50 of 2.49 Mb. Ninety percent of the genome assembly was included in the 1,091 longest super-scaffolds (Table 1 above). The overall size of the hamster genome was estimated to be 2.7 Gb using the k-mer estimation method (FIG. 5). Optical mapping data were further combined with published BAC-based fluorescence in situ hybridization data (Cao et al. Biotechnology and bioengineering 109, 1357-1367 (2012)) to successfully associate 26% of the genome sequence data to specific hamster chromosomes (Supplementary Tables 3 and 4, publicly available on the world wide web at nature.com/nbt/journal/v31/n8/full/nbt.2624.html#supplementary-information).

To assess the coverage of the hamster transcripts in the assembly, mRNA was sequenced from a pool of hamster tissues and de novo assembled the transcriptome into 98,116 contigs (Methods). Mapping RNA-seq contigs to the genome assembly demonstrated that >90% of the assembled transcripts could be associated with annotated genes (Supplementary Table 5, publicly available on the world wide web at nature.com/nbt/journal/v31/n8/full/nbt.2624.html#supplementary-information).

Genome Annotation

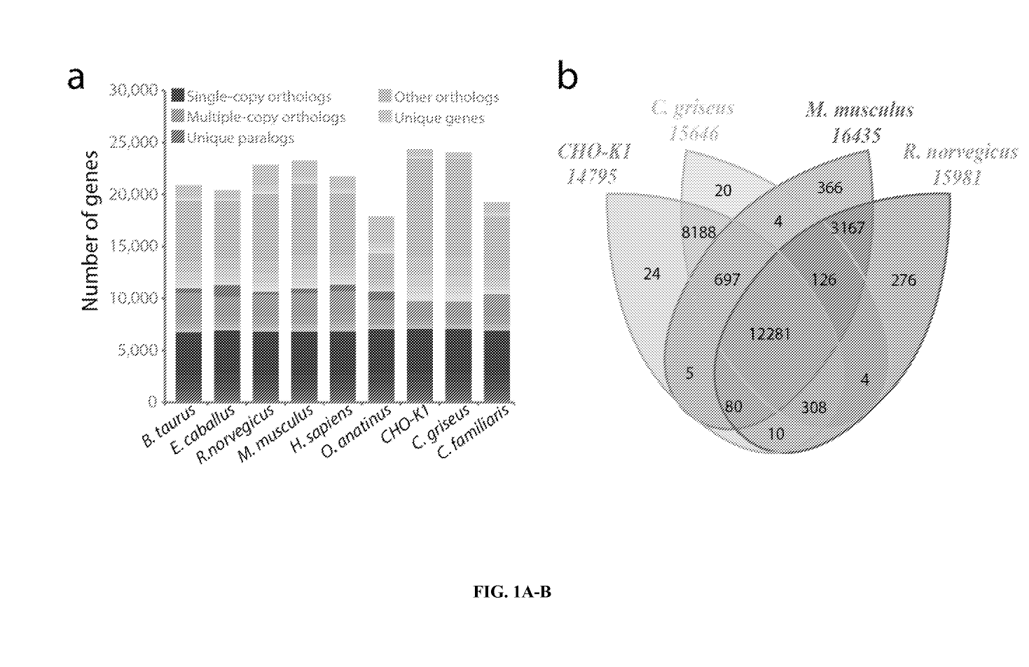

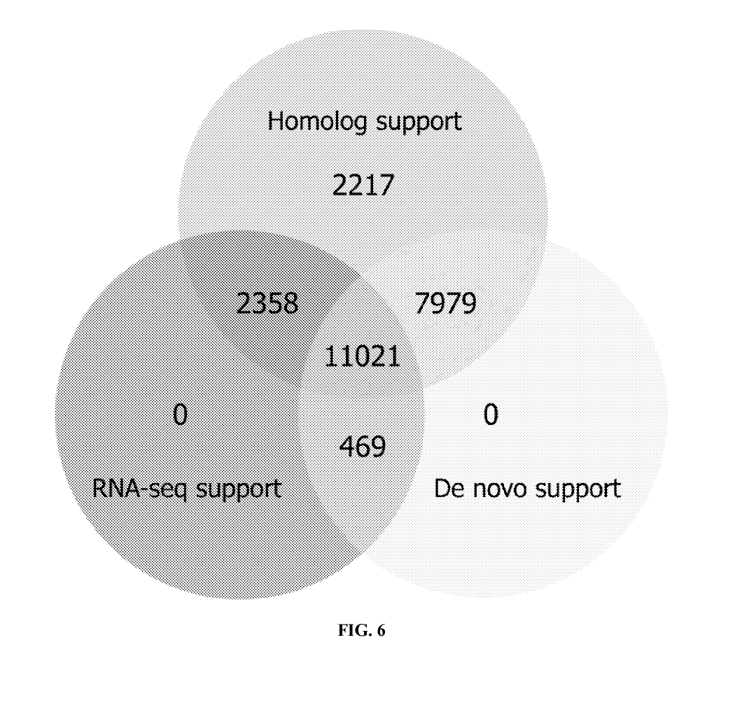

Repeat features were analyzed and endogenous retroviral elements identified (Supplementary Notes below and Supplementary Tables 6-9, publicly available on the world wide web at nature.com/nbt/journal/v31/n8/full/nbt.2624.html#supplementary-information). Genes were predicted using homology-based approaches, de novo gene prediction algorithms and transcriptome-based methods (FIG. 6 and Supplementary Table 10, publicly available on the world wide web at nature.com/nbt/journal/v31/n8/full/nbt.2624.html#supplementary-information). The final gene set consisted of 24,044 genes in the hamster genome, which is similar to that of the CHO-K1 cell line. See, Xu et al. Nature Biotechnology 29, 735-741 (2011). Of these predicted genes, 23,473 clustered into 21,628 gene families (FIG. 1a), and 3,052 (14.1%) gene families contained more than one gene in the hamster. Only 20 gene families were unique to the hamster, when compared to the rat, mouse and CHO-K1 genomes (FIG. 1b). 82% (19,775) of the predicted genes were functionally annotated using InterPro™, Swiss-Prot™, TrEMBL™, Gene Ontology™ (GO) and KEGG™ (Supplementary Table 11, publicly available on the world wide web at nature.com/nbt/journal/v31/n8/full/nbt.2624.html#supplementary-information).

Comparison Between Hamster and CHO-K1 Genomes

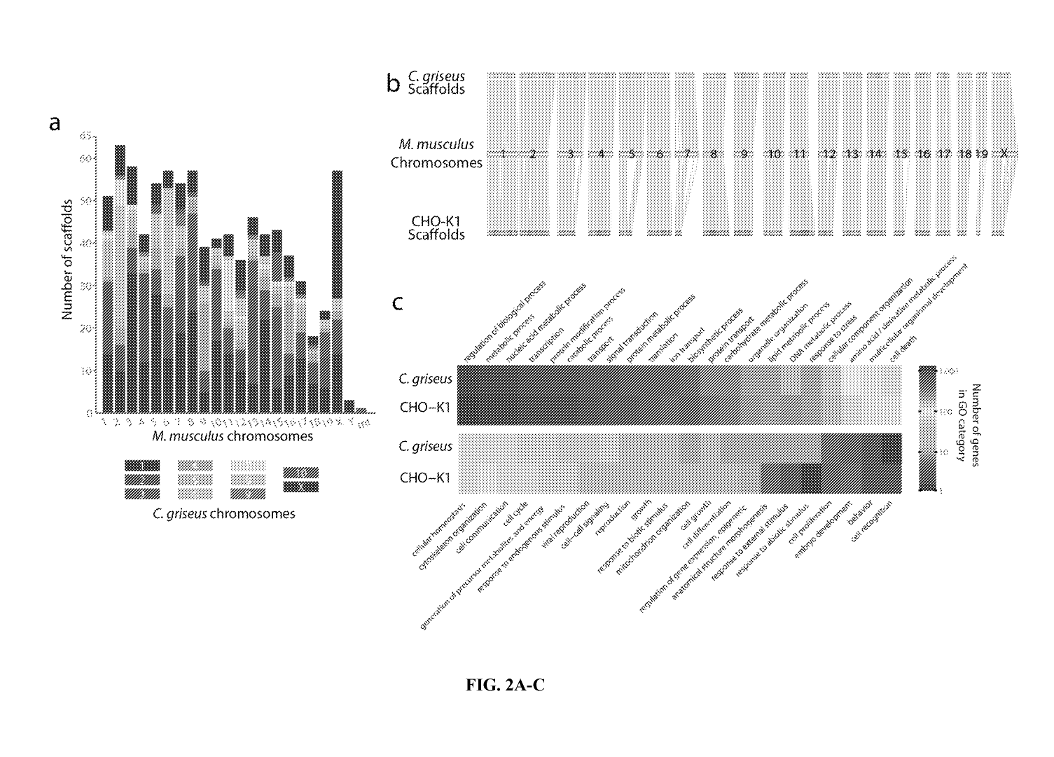

Mutations and structural variations are common in mammalian cell line genomes. Although large chromosomal rearrangements have been shown in CHO cell lines previously, the extent of these changes at the sequence level remains unknown. Thus, the structure and gene content of the Chinese hamster genome and the published genome of CHO-K1 cells from the American Type Culture Collection™ (ATCC) (Xu, X. et al. The genomic sequence of the Chinese hamster ovary (CHO)-K1 cell line. Nature Biotechnology 29, 735-741 (2011)) were compared. To facilitate this comparison, all large hamster and CHO-K1 scaffolds were aligned to the mouse chromosomes. Numerous chromosomal translocations have occurred through evolution since the mouse and hamster diverged (FIG. 2a). However, no large sections of the mouse chromosomes were missing in the hamster (FIG. 2b). On the other hand, CHO-K1 scaffolds failed to align to portions of mouse chromosomes 5, 7, 15 and 19 (FIG. 2b). Meanwhile, Illumina sequencing reads from CHO-K1 aligned to the hamster scaffolds corresponding to these regions. This result suggests the possibility that these regions are in CHO-K1, albeit considerably mutated or rearranged. Next the scope of mutations was directly assessed by comparing the CHO-K1 genome to the hamster genome. CHO-K1 contained 25,711 structure variations, including 13,735 insertions and 11,976 deletions (Supplementary Notes below and Supplementary Table 12, publicly available on the world wide web at nature.com/nbt/journal/v31/n8/full/nbt.2624.html#supplementary-information). Despite the large number of structural variations in CHO-K1, the set of annotated genes in the hamster and CHO-K1 were highly similar. Specifically, there was a 99% overlap in gene content between the two genomes, and an assessment of GOslim terms for these genes further reiterated the similarity in gene content (FIG. 2c).

Variation Between Different CHO Cell Lines

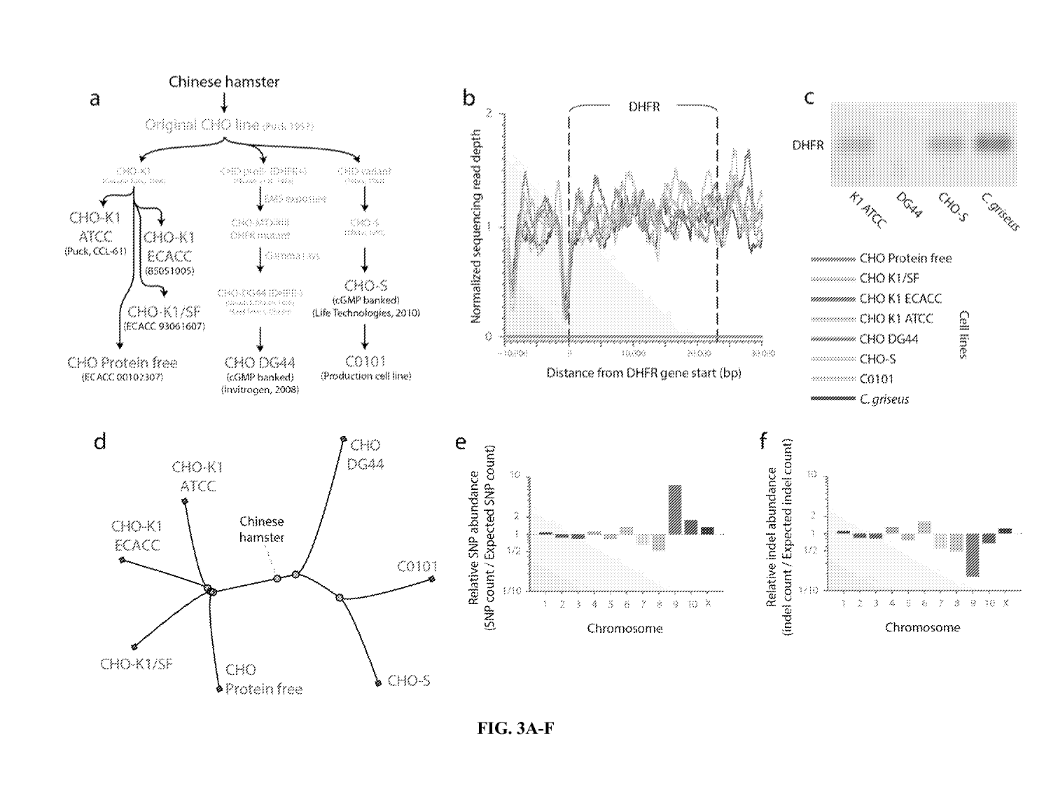

Despite the similarity in gene content, numerous genomic variations were detected in CHO-K1 relative to the hamster. To elucidate the extent of genomic heterogeneity across other cell lines, six additional CHO cell lines were sequenced (FIG. 3a) to >9× depth, covering ˜95% of each genome. Including the previously sequenced CHO-K1 genome, the seven cell lines accounted for three different lineages and several different phenotypic features, e.g., cells adapted to different media, suspension-grown cells, antibody-producing cells, etc. (Supplementary Table 13, publicly available on the world wide web at nature.com/nbt/journal/v31/n8/full/nbt.2624.html#supplementary-information).

To initially validate the cell line resequencing data, the genotype related to an important phenotypic marker for CHO cell lines was inspected. Certain cell lines lack dihydrofolate reductase (DHFR) activity, and cannot grow without glycine, hypoxanthine and thymidine (GHT). However, when an exogenous DHFR gene is coupled to a desired protein product gene on the same plasmid, the GHT media and methotrexate can be used to select for clones that over-produce DHFR and the recombinant protein of interest. Among the cell lines sequenced here, only the DG44 cell line is known to carry the DHFR-phenotype. Consistent with this characteristic, all cell lines had genomic sequence data for the DHFR gene, except for DG44 (FIG. 3b). This DG44-specific deletion was further confirmed by PCR (FIG. 3c).

To assess the genome-wide differences between these CHO cell lines, the hamster genome was utilized as the reference sequence. This reference sequence allowed determination of single nucleotide polymorphisms (SNPs), short insertions and deletions (indels) and gene copy number variations (CNVs) (Supplementary Table 14, publicly available on the world wide web at nature.com/nbt/journal/v31/n8/full/nbt.2624.html#supplementary-information). Across the cell lines, 3,715,639 SNPs were identified, and a phylogenetic reconstruction based on these SNPs accurately recapitulated the cell line history (FIG. 3d). Also 551,240 indels shorter than 5 bp, 319 were identified of which are predicted to be frame-shifting indels in coding regions. SNPs and indels did not occur uniformly, and some hamster chromosomes were more affected than others (FIGS. 3e and 3f).

3,383 non-redundant duplicated regions in at least one cell line were found and 177 duplicated regions in all seven cell lines were found (Supplementary Table 15, publicly available on the world wide web at nature.com/nbt/journal/v31/n8/full/nbt.2624.html#supplementary-information). In total, 4,241 genes resided entirely within these 3,383 duplicated regions. Moreover, 113 genes were found to have a reduced copy number in one or more cell lines. In addition, 17 hamster genes were completely missing in at least one cell line, and the missing genes often differed between the lineages (Supplementary Table 16, publicly available on the world wide web at nature.com/nbt/journal/v31/n8/full/nbt.2624.html#supplementary-information).

A variety of genes are associated with mutations and CNVs (Supplementary Tables 17-20, publicly available on the world wide web at nature.com/nbt/journal/v31/n8/full/nbt.2624.html#supplementary-information). Of the SNPs, 5,487 (0.15%) were nonsynonymous and significantly enriched in many GO classes (FDR<0.01), such as olfactory genes and GPCRs (P<2×10⁻²⁵ and 6×10⁻²¹, respectively; hypergeometric test), whereas genes in these same classes were rarely duplicated (P<1×10⁻⁵ and 0.02, respectively; hypergeometric test). In addition, proteins involved in cell adhesion were also enriched in SNPs (P<0.004; hypergeometric test). It is possible that these mutations allow CHO cells to grow in suspension cultures without adhesion factors.

Other genes were protected from SNPs, such as genes associated with DNA binding and transcription regulation and metabolism (P<0.006 and P<9×10⁻⁵, respectively; hypergeometric test). Notably, some signaling pathways were insulated from SNPs, such as the WNT and mTOR signaling pathways (P<0.02 and P<0.002, respectively; hypergeometric test) and autophagy (P<0.01). These pathways all contribute to the proliferative and immortalized phenotypes in cancer cells and likely play a similar role in CHO cell lines. Protein glycosylation was also significantly insulated from SNPs in all cell lines (mean hypergeometric P=0.018). Thus, the distribution of mutations and CNVs seems consistent with traits that make CHO cell lines desirable protein production hosts (i.e., high proliferation rate, suspension growth and protected protein glycosylation).

Using the Genome to Study the Apoptosis Pathway

CHO production strains can be grown to high cell densities in fed-batch cultures with serum-free media. Bioprocessing limitations in nutrients in these environments can lead to apoptosis, thereby limiting viable cell density and volumetric productivity. To improve bioprocessing efficiencies, many researchers have sought to improve cell line longevity by suppressing apoptosis in CHO cells. These efforts involve modulating protein activity by over-expressing anti-apoptotic pathways and blocking pro-apoptotic pathways with chemicals, siRNA and gene deletions. However, the complex nature of apoptosis has made it non-trivial to optimize in CHO cells. Thus, a more complete view of gene expression and mutations in the apoptosis system could facilitate bioprocessing and cell engineering efforts to control cell death.

First homologs for anti- and pro-apoptotic proteins in the C. griseus genome were identified to assess changes in apoptosis in CHO cells (Supplementary Table 21, publicly available on the world wide web at nature.com/nbt/journal/v31/n8/full/nbt.2624.html#supplementary-information). Of the 62 KEGG™ orthologous gene identifiers in apoptosis, 92% were in the hamster genome. Consistent with observations in mouse, caspase-10 was missing. Other missing genes included interleukin-3, interleukin-3 receptor alpha and interleukin-1 alpha. Although these genes were undetected, apoptosis utilizes redundant pathways, and the lack of these genes should not hinder the system.

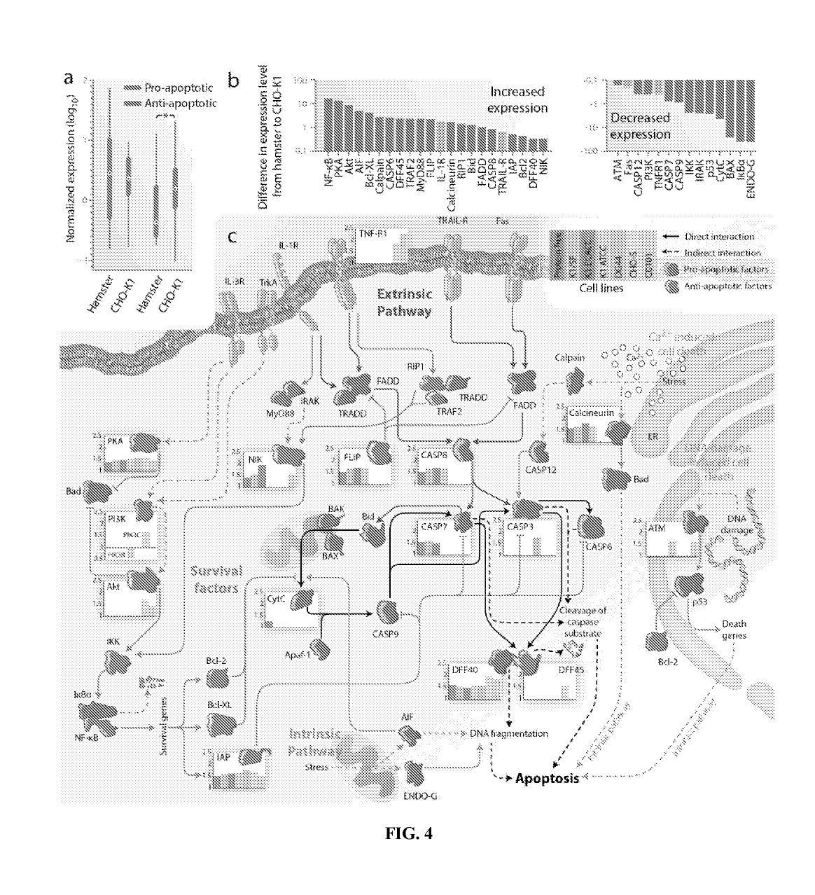

In the CHO-K1 cell line, no additional genes for anti- and pro-apoptotic proteins were lost relative to the hamster. Instead, apoptotic gene expression significantly changed. Pro-apoptotic genes exhibited slightly lower gene expression in CHO-K1 in comparison to C. griseus, although this was not statistically significant. However, anti-apoptotic genes in CHO-K1 exhibited significantly higher median expression (P<0.02; Wilcoxon rank-sum test; FIG. 4a). Apoptotic genes with the greatest increase in expression tend to be anti-apoptotic (e.g., NF-κB, protein kinase A, Akt and Bcl-XL), whereas repressed genes tend to be pro-apoptotic (e.g., endonuclease G, IκBα, BAX, and p53) (FIG. 4b). Thus, CHO-K1 suppresses apoptosis, and it is anticipated that similar gene expression changes occur in other CHO cell lines.

In addition to changes in apoptotic gene expression, CNVs also frequently occur in apoptotic genes in mammalian cell lines. Since CNVs can complicate efforts to engineer cell lines, CHO CNVs in the context of the apoptosis pathways was also analyzed.

The apoptotic network is stimulated by external signals through the extrinsic pathway, or internal stress signals (e.g., increases in cytosolic Ca²⁺ or DNA damage) through the intrinsic pathway. The diverse signals transmitted by each pathway converge upon the caspase proteases, which cleave protein targets and lead to cell death. As a strategy to increase CHO cell longevity, caspase activation has been targeted with chemical inhibitors and caspase-inhibiting proteins. It was found that several cell lines contained extra copies of caspase (FIG. 4c). Thus, efforts to remove pro-apoptotic genes, such as caspases, should account for potential CNVs for those genes. Some anti-apoptotic genes were only duplicated in individual cell lines, which may lead to these lines being more resilient against apoptosis activation. For example, the Inhibitors of Apoptosis (IAP) family of proteins inhibit caspases, and one IAP gene, BIRC7, was found to be duplicated in all cell lines. In addition, another anti-apoptotic factor, phosphoinositide 3-kinase (PI3K), also showed cell-type specific CNVs.

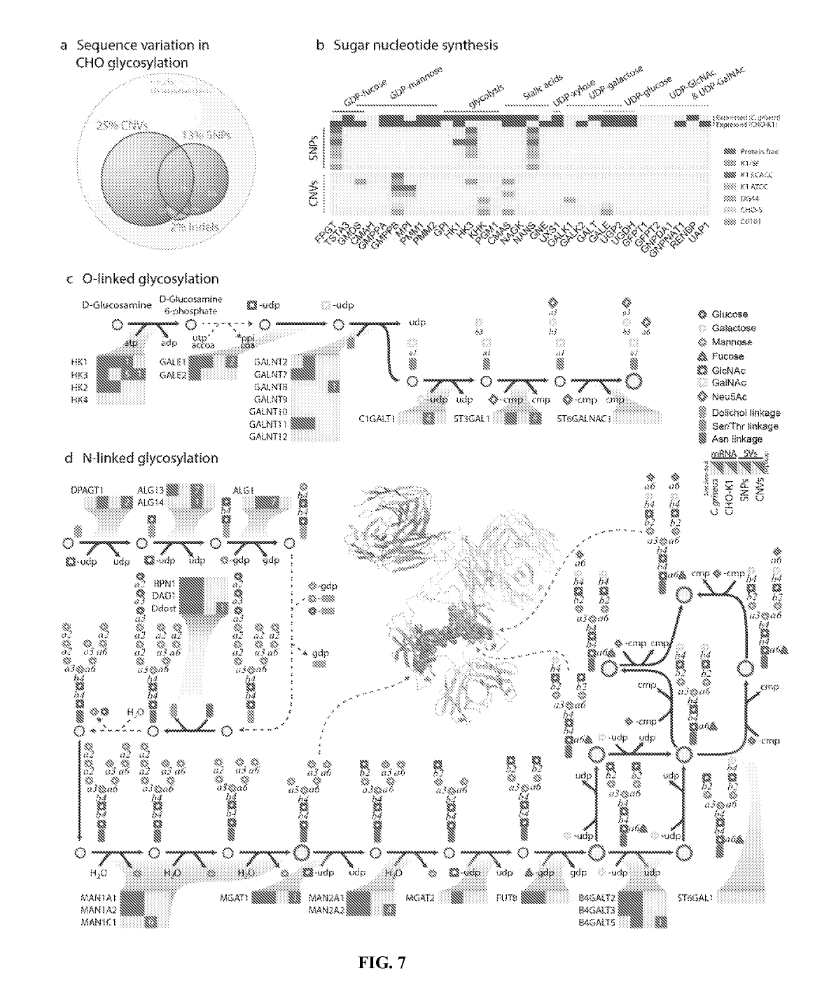

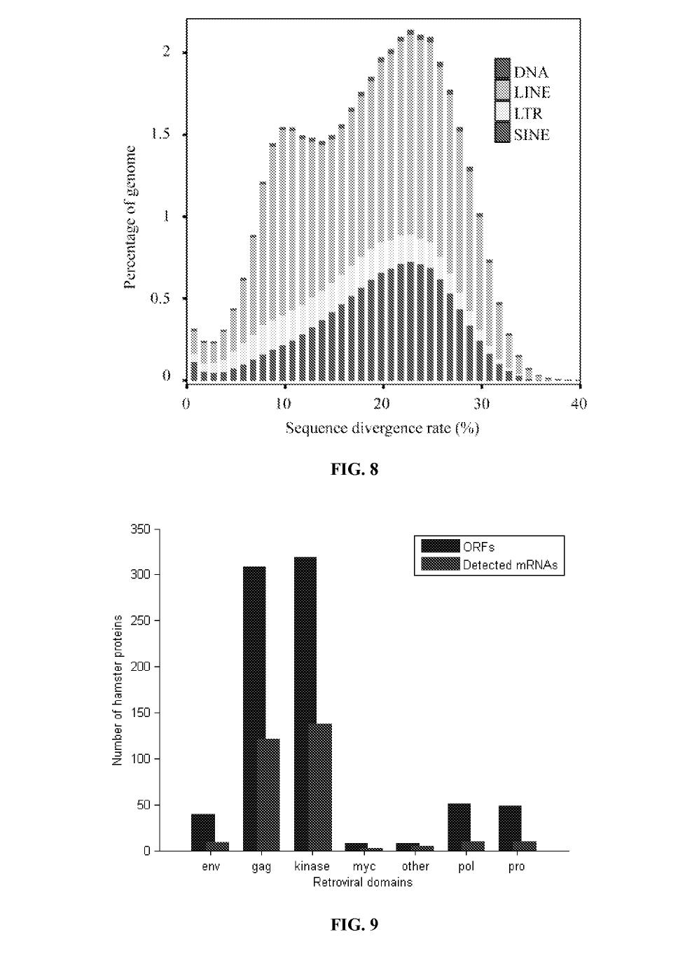

In general, CNVs occur in various pathways, such as apoptosis and glycosylation (FIG. 7) and can differ between cell lines (Supplementary Table 22-23, publicly available on the world wide web at nature.com/nbt/journal/v31/n8/full/nbt.2624.html#supplementary-information). Knowledge of CNVs can help researchers avoid unexpected genomic changes when using nucleases in duplicated regions. CNVs can be clone-specific as gene copy numbers in a single cell line vary considerably during growth media adaptation or after several cell passages. Thus, clone-specific genomic data may indicate which cell line modifications will be effective for a particular production cell line under development.

### DISCUSSION

Genomic resources have provided a wealth of tools in biotechnology, ranging from phenotyping tools, such as transcriptomics, to genome editing technologies. These resources have transformed our ability to study and modify the functions of human cells (e.g., cancer and HEK cells) and other model organisms. Similar tools are becoming available for CHO cells, but maximizing their potential requires a clear picture of the genomic landscape of CHO cells. Herein it is demonstrated how the C. griseus genome can provide a sequence-level view of genomic heterogeneity between cell lines and yield a more comprehensive picture of the variants in a cell line of choice.

Numerous studies have shown large chromosomal rearrangements in CHO cells, using banding techniques and fluorescence in situ hybridization. These approaches identified large translocations in CHO cells, providing a coarse-grained view of genomic variations in these unstable genomes. For the first time, a whole-genome sequence-level view of the heterogeneity between CHO cell lines is presented. It is shown that each cell line harbors a unique set of mutations, including SNPs, indels, CNVs and missing genes. CNVs were particularly heterogeneous, with 48% (mostly duplications) being unique to one cell line (Supplementary Table 15, publicly available on the world wide web at nature.com/nbt/journal/v31/n8/full/nbt.2624.html#supplementary-information). It was also found that mutations rapidly accumulate during production cell line development. For example, during the development of the C0101 antibody-producing cell line from CHO-S, 301,753 new SNPs arose, representing 9% of the SNPs in that cell line.

The non-uniform distribution of mutations in each cell line seemed to have some phenotypic relevance. Indeed, several processes associated with proliferation and immortalized phenotypes were more insulated from mutation. These included the WNT and mTOR signaling pathways and processes such as autophagy. Mutations in other pathways such as glycosylation and viral susceptibility (Supplementary Notes below) varied between cell lines and might influence desired phenotypic properties, although careful biochemical studies are needed. Duplications were also seen for many apoptotic genes. Notably, many of the sequence variations were shared between members of the same family of CHO cells (i.e., CHO-K1, DG44 or CHO-S), but these were frequently not shared across CHO cell families (Supplementary Tables 16, 22-24, publicly available on the world wide web at nature.com/nbt/journal/v31/n8/full/nbt.2624.html#supplementary-information). Moving forward, a detailed knowledge of mutations in each cell line may be valuable for cell line selection, characterization and engineering, as well as bioprocess and media optimization, and cell line characterization. This knowledge for each cell line may further improve the success of siRNAs, zinc finger nucleases and other cell line engineering tools. Additionally, as more sequence variation data are collected on diverse cell lines, it may be possible to associate cell phenotypes with different mutations (as is commonly done in model organisms).

To fully detail the sequence variations, it is necessary to have a well-defined reference genome with relevance to all CHO cell lines. The reference genome should exhibit several properties. First, it must contain the genomic sequence of all native CHO genes and their regulatory elements. It was found that CHO-K1 seems to be missing certain hamster genes, and that cell lines from other lineages are missing other genes (Supplementary Table 16, publicly available on the world wide web at nature.com/nbt/journal/v31/n8/full/nbt.2624.html#supplementary-information). Although the focus was on genes that are entirely missing, many more truncated genes and disrupted promoter elements may be found in each cell line as gene models are improved and as regulatory elements are discovered.

Second, it is often desirable to identify all variants in a cell line, and not just the genomic differences between two cell lines. There are clear ultra-structural differences between the hamster and CHO cells, although some translocations are conserved among cell lines. These structural variations are likely conserved because CHO cells from the CHO-K1, DG44 and CHO-S lineages share a common highly mutated ancestor. Indeed, it was found that 67% of SNPs (˜2.5 million) were shared among all CHO cell lines. These shared variants would be missed if the CHO-K1 genome were used as the sole reference. Mutations with deleterious effects on expression and/or activity can be more comprehensively cataloged using the hamster genome as the reference. Thus, endemic loss-of-function mutations in CHO could be identified and remedied as needed for a desired phenotype.

Third, a reference genome must be amenable to improvement over time. The chromosomes of CHO cell lines are unstable, with non-negligible karyotypic differences even in the same culture. Thus, it will be much easier to develop and maintain a gold standard reference sequence of the more stable Chinese hamster genome. This resource will be valuable for characterizing CHO cell lines and using -omic technologies, akin to how the M. musculus genome is used for studying murine cell lines. Furthermore, although regulatory challenges remain for cell line engineering, whole-genome resequencing against a reference genome will provide transparency as regulatory agencies assess products from engineered cell lines for approval.

CHO cell lines exhibit important differences in genomic content that can influence cell line traits. These are likely to be further extenuated by differences in gene expression levels. As a result, genome-scale viewpoints will likely become increasingly relevant for CHO based bioprocessing, as they have for microbe-based manufacturing over the past decade. Although these approaches can require expensive phenotyping and -omic technologies, costs are rapidly decreasing. Thus, genome-scale analyses may enhance our ability to understand the production characteristics of CHO cell lines and aid in the production of therapeutic proteins in the coming decades.

Supplementary Notes

Filtering of Raw Reads Prior to Assembly

Illumina® reads were filtered based on following criteria.

Reads were filtered when more than 10% of the bases were degenerate (N) or poly-A's.

Reads were filtered if they were from large insert size libraries (2 kb, 5 kb, 10 kb and 20 kb) with 15 or more bases having phred quality score (given by Illumina™ sequencer) less than or equal to 7, or from short insert size libraries with 50 bases having quality score less than or equal to 7.

Reads were filtered if more than 10 bp aligned to the adapter sequence.

If read pairs in which read 1 and read 2 overlapped more than 10 bp (allowing 10% mismatch), they were filtered.

Reads were filtered if they were PCR duplicates (i.e., two completely identical reads).

Using these criteria, 347.5 Gb of raw data was filtered to 240.5 Gb representing ˜90× coverage of the hamster genome.

Estimation of Genome Size

Through k-mer estimation, the genome size was found to be 2.7 Gb (FIG. 5), which is smaller than previous estimates using Feulgen densitometry (3.05 Gb˜3.99 Gb), but slightly larger than the k-mer estimate of Chinese hamster ovary (CHO)-K1 cell line, which was 2.6 Gb (FIG. 5).

Repeat Features in the Chinese Hamster Genome

Repeat elements in the genome were predicted using several methods. Tandem repeats were first predicted using RepeatMasker™ and TRF™. Then transposable elements (TEs) were identified using a combination of homology-based and de novo methods. For the homology-based method, databases of known repetitive sequences were used in Repbase™ and searched against the hamster draft genome using RepeatMasker™. For the de novo method, three software packages were used, including LTR_FINDER™ (Version 1.0.3), PILER™ and RepeatScout™ (Version 1.05), to build a de novo repeat database of the Chinese hamster. Then RepeatMasker™ (Version 3.2.7) was used to identify repeats and used RepeatProteinMask™ (available on the world wide web at repeatmasker.org/, Version 3.2.2) to search the protein database in Repbase™ against the genome to identify repeat-related proteins. Finally the de novo prediction and the homolog prediction of TEs were combined according to the position in the genome. It is estimated that repeat elements account for 42.8% of the genome (Supplementary Table 6, publicly available on the world wide web at nature.com/nbt/journal/v31/n8/full/nbt.2624.html#supplementary-information). Specifically, ˜41% of the genome consists of transposable elements (TEs) (Supplementary Table 7, publicly available on the world wide web at nature.com/nbt/journal/v31/n8/full/nbt.2624.html#supplementary-information), which is similar to that of the mouse (37.5%) (Waterston et al. Nature 420, 520-562 (2002)) and rat (40%) (Gibbs et al. Nature 428, 493-521 (2004)). The most abundant TE, long interspersed repeated DNA (LINE), accounts for 27% of the genome. The distribution of sequence divergence rate for LINE elements was bimodal suggesting that two independent burst events of LINEs may have occurred within the hamster genome (FIG. 8). Since other TEs, such as long terminal repeats (LTRs) and short interspersed repeated DNAs (SINEs), only have one peak, these might not have experienced the recent burst events as seen with LINEs. Further the detailed categories of different TEs (Supplementary Table 8, publicly available on the world wide web at nature.com/nbt/journal/v31/n8/full/nbt.2624.html#supplementary-information) were analyzed and compared to mouse, rat and human. The composition of TEs in hamster genome is similar to those of rat and mouse.

Endogenous Retroviral Sequences in the C. griseus Genome

Consistent with other mammalian genomes, the C. griseus genome has many stretches of DNA with homology to viral genes. This is of particular interest in C. griseus since CHO cell lines have shown substantial resistance to many human viruses, and they do not seem to produce infectious retroviruses as seen in other rodent cell lines. However, numerous reports have observed viral particles budding off of CHO cells. Since these viral particles likely represent endogenous viral proteins as opposed to new viral infections, it would be of interest to assess the origin of these viral particles. Here a preliminary identification of endogenous viral elements in the Chinese hamster genome is provided.

Two classes of viral particles have been observed in CHO cell lines. Type A particles are immature intracellular particles that are derived from endogenous retroviral-like genes. Genes coding these particles often lack a functional env gene and therefore are unable to infect other cells, but instead behave like retrotransposons and readily spread through the host genome. Type A particles have been previously identified and their associated genes have been sequenced in Syrian hamster and mouse. These sequences were used to identify similar RNAs in a CHO-K1 derivative cell line. However, none of the identified sequences could encode functional proteins since they all contained premature stop codons or frameshift mutations. Similarly, budding type C particles have also been observed. When RNA was isolated and sequenced from these particles, it was found that these are also unable to encode functional proteins due to numerous mutations. Thus it is still unclear where these particles are encoded in the Chinese hamster and its derivative cell lines.

The existence of C-type particles suggests that full-length coding retroviral proteins should still exist. Thus, to gain a preliminary view of the landscape of endogenous retroviral elements, a list of 115 sequenced retroviral genomes was compiled and genes that represent potentially intact viral proteins encoded by the hamster genome were searched. This was done by conducting a blastp query of all retroviral proteins against all putative protein-coding ORFs in the hamster. With an E-value cutoff of 1×10⁻⁶ 403 proteins in the hamster genome were found with homology to 174 proteins in the 115 retroviral genomes. These 174 viral proteins contained the domains common to retroviruses (FIG. 9). Furthermore, it was found that 80% of the 403 hamster proteins were at least as long as their retroviral homologs and 40% of their mRNAs were expressed, thereby suggesting that many these may be still be synthesizing retroviral components. Protein sequences for all of the ORFs with homology to retroviral proteins are provided in Supplementary Table 9, publicly available on the world wide web at nature.com/nbt/journal/v31/n8/full/nbt.2624.html#supplementary-information.

Properties of Scaffold Chromosome Assignment

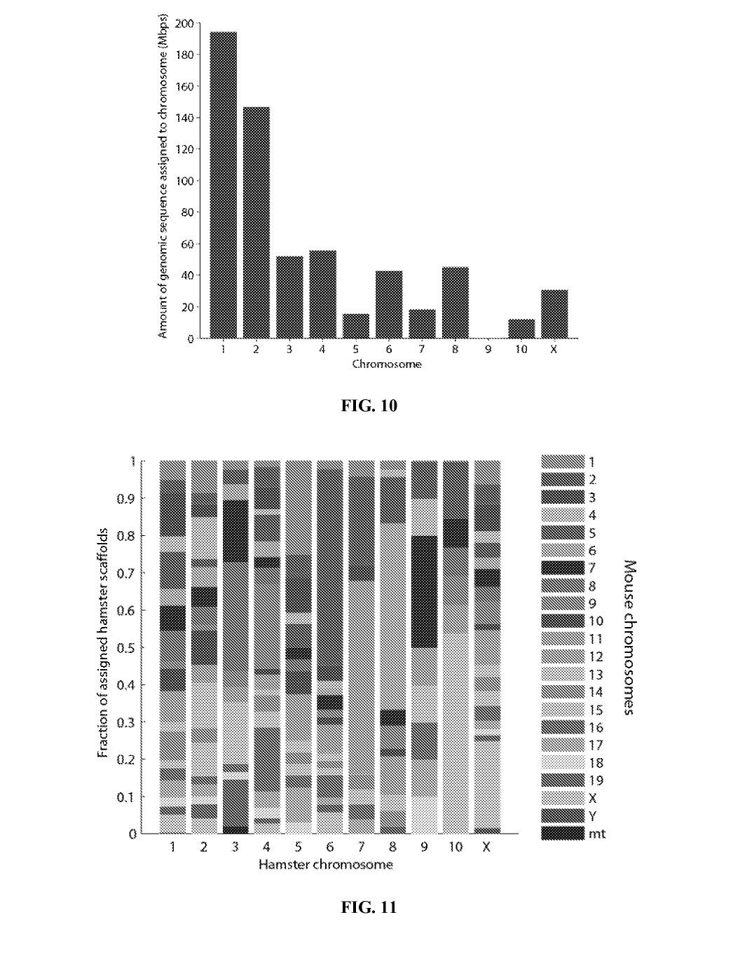

To assign scaffolds to their respective chromosomes, our optical mapping data were used in conjunction with published BAC end sequencing and fluorescence in situ hybridization. Specifically, chromosomal assignments were obtained for each BAC, and then blastn was used to find scaffolds with the highest homology to BAC end sequences (E-value<1×10⁻⁵). Once chromosomal assignments based on the BAC library were obtained for scaffolds (Supplementary Table 3, publicly available on the world wide web at nature.com/nbt/journal/v31/n8/full/nbt.2624.html#supplementary-information), the chromosomal localization was inferred for all scaffolds that were joined to previously assigned scaffold by optical mapping (Supplementary Table 4, publicly available on the world wide web at nature.com/nbt/journal/v31/n8/full/nbt.2624.html#supplementary-information). From this analysis, 26% of the genomic sequence to hamster chromosomes (FIG. 10) was loalized. Hamster scaffolds were aligned to the Mus musculus genome to assess the extent to which the species had diverged (FIG. 11).

Details of the Comparison Between C. griseus and CHO-K1

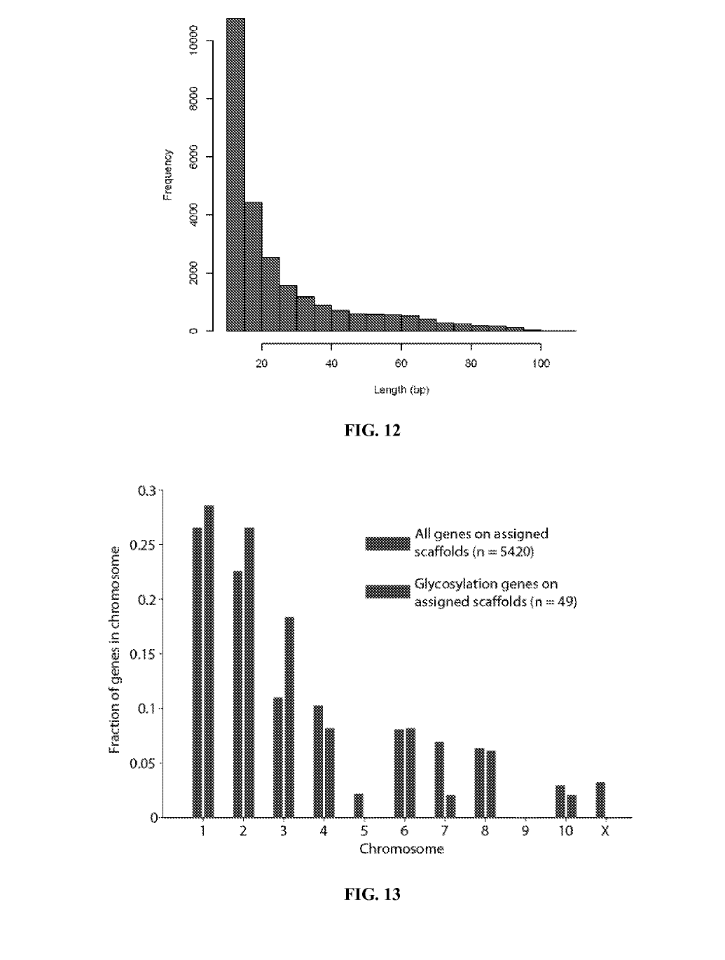

The hamster and CHO-K1 genome sequences were compared and 25,711 structure variations were identified including: 13,735 insertions and 11,976 deletions in CHO-K1, relative to the hamster. Most of these variations are shorter than 100 bp (FIG. 12). These variations were observed in 2,188 genes in the hamster genome and were enriched in genes with binding function, which might have changed during the cell line formation (Supplementary Table 12, publicly available on the world wide web at nature.com/nbt/journal/v31/n8/full/nbt.2624.html#supplementary-information).

A comparison of the gene content of the two genomes showed that 99% of the hamster genes (24,021) had homologs in CHO-K1 with over 60% identity and 60% coverage. Similarly, 24,109 of the 24,383 (99%) CHO-K1 genes had homologs in the hamster genome with over 60% identity and 60% coverage.

Chromosomal Localization of Glycosylation Enzymes in C. griseus and CHO-K1

One beneficial trait of Chinese hamster ovarian cell lines is their ability to glycosylate enzymes in a fashion that is compatible to humans. Thus, since one may desire to genetically modify CHO cell lines in order to improve product titer, it is desirable to know where the glycosylation enzymes reside, so that care can be taken to avoid unintended disruption of the glycosylation pathways. For each glycosylation enzyme (Supplementary Table 26, publicly available on the world wide web at nature.com/nbt/journal/v31/n8/full/nbt.2624.html#supplementary-information), the scaffold upon which it is located was identified, allowing one to design primers to verify that desired glycosylation enzymes are not disturbed in cell engineering. Furthermore, using the BAC and optical mapping data, 26% of the genomic sequence data was associated to specific chromosomes. This allowed us to assign 49 glycosylation enzymes to specific hamster chromosomes. It is noted that the distribution of these glycosylation enzymes across all chromosomes roughly reflects the distribution of genes in general. Indeed, they were not significantly enriched or depleted from any one chromosome (p>0.1; FIG. 13).

Copy Number Variations in Anti-Apoptotic Genes

Many proteins balance the pro-apoptotic activities, and when over-expressed, these proteins can inhibit apoptosis in CHO cells. Different cell lines may naturally be less apoptotic, since some anti-apoptotic genes were duplicated in individual cell lines. For example, IAP genes inhibit caspases, and one gene, BIRC7, was found duplicated in all cell lines.

While several anti-apoptotic genes have increased copy numbers in CHO cell lines, it was found that an upstream factor, phosphoinositide 3-kinase (PI3K) showed cell-type specific responses. The anti-apoptotic PI3K genes encode catalytic (PIK3C) and regulatory (PIK3R) subunits. Together, these proteins relay survival signals to proteins such as protein kinase B (Akt). Few sequence variants were detected in PI3K, but it was found that CHO-S had a duplication of the catalytic subunit, and CHO-K1 had a deletion of the regulatory subunit. These differences in copy number may influence apoptotic activity in the cell lines with CNVs.

Mutations in Sugar Nucleotide Synthesis and Glycosylation

Protein glycosylation significantly influences biotherapeutic quality. Glycosylation can vary substantially between different parent organisms and cell lines, and is also influenced by mutations, culture conditions, and enzyme expression levels. Although there are fewer SNPs in glycosylation than expected by chance (mean p=0.018), there are still many mutations. To gain a more detailed view of mutations in glycosylation, a bidirectional blastp was conducted between the human genome and CHO-K1 glycosylation genes. Among 256 enzymes associated with glycosylation in the hamster, 13%, 2% and 25% have non-synonymous SNPs, frame-shifting indels, or CNVs, respectively, in at least one cell line (FIG. 7a and Supplementary Tables 23-24 and 26, publicly available on the world wide web at nature.com/nbt/journal/v31/n8/full/nbt.2624.html#supplementary-information).

Sugar nucleotides are the building blocks for glycans. Transcripts for most sugar nucleotide synthesis enzymes are detected in the hamster and/or CHO-K1. However, between cell lines, there may be variations in sugar nucleotide abundance since most synthesis pathways have a mutation or CNV (FIG. 7b). Next in glycosylation, these sugars are sequentially added to growing oligosaccharide chains. To study these pathways, human glycosylation reactions associated with the bidirectional hits using the human metabolic network, Recon 1 were determined. The reactions were then cross-referenced with glycoslyation reactions required for producing N-glycans and O-glycans found on common IgG. It was found that mutations and CNVs occur at various locations in the pathways synthesizing different glycans, including N- and O-glycans (FIGS. 7b-7d). While not all SNPs will have a measurable effect on enzyme activity or substrate preference, these glycans are produced through long, branching pathways. Thus, there is a considerable increase in the probability of having a mutation that changes the final glycoform in any given cell line. Furthermore, a few cell lines have CNVs in the FUCA1 fucosidase gene, α(2,3)-sialyltransferase (ST3GAL1), and the 3′-phosphoadenosine-5′-phosphosulfate transporter SLC35B3. These genes have important functions in removing fucose, adding sialic acid, and modulating sulfation, respectively. The processes to which these genes contribute also have been shown to affect the activity and longevity of recombinant proteins. The heterogeneity in glycosylation enzyme mutations across different cell lines could have dramatic effects on the protein glycoforms produced in different cell lines, yielding differences in in vivo product activity and clearance rates of recombinant therapeutic proteins. Thus, it will be important to assess the mutations, copy numbers and expression levels of these and other enzymes in production cell lines in order to optimize the modifications added to recombinant proteins.

Viral Susceptibility

The resistance of CHO cell lines to viral infection is a beneficial trait from a bioprocessing perspective; thus, the hamster and cell line genomes were examined for mutations or changes in gene expression that may influence viral susceptibility (Supplementary Table 27, publicly available on the world wide web at nature.com/nbt/journal/v31/n8/full/nbt.2624.html#supplementary-information). Many viral susceptibility genes were previously shown in CHO-K1 to be not expressed, and many of these were key viral entry receptors. It was found that 70% of the viral susceptibility genes were expressed in the hamster transcriptome, which is more than expected by chance (p=3×10⁻³⁹), while only 55% of the viral susceptibility genes were expressed in CHO-K1, despite having more expressed transcripts across the entire CHO-K1 genome³. Non-expressed viral susceptibility genes in CHO-K1 were enriched in non-synonymous SNPs (p=0.02). Interestingly, 28 of these 33 SNPs in viral susceptibility genes were shared across all cell lines. These included plasma membrane receptors and viral entry receptors, such as integrins (ITAV, ITAX, ITAM), CD4, and CD86. While the hamster expresses many viral entry receptors, it seems that CHO cell lines have managed to down-regulate or mutate many viral susceptibility genes, thereby contributing to this favorable bioprocessing trait for CHO cell lines.

### Example 2

### Metabolic and Glycosylation Model Construction

A genome-scale constraint-based metabolic model of C. griseus, the Chinese hamster, was created based on the genome sequence and annotation (Example 1) and the human Recon 2 model (Thiele et al. Nature Biotechnology 31(5):419-25 (2013)) followed by manual curation. Reactions were removed from Recon 2 when they were carried out by genes not present in the C. griseus genome. Additional reactions were added when required to run computations using the model. The resulting C. griseus model has been used to create C. griseus derived cell line models by using experimental data collected for these cell lines. Specifically this was done for the cell line CHO-K1 and CHO-S using RNAseq to determine the presence of genes, but could be used to create any C. griseus derived cell line model. These cell line models have been validated using measurements of external metabolites as inputs to constrain the model, and then growth rate predictions were made. The human Recon 2 model contains 2194 genes, each associated with one or more reactions totaling 3919 gene associated reactions. The model additionally contains 3522 non gene associated reactions representing for example unknown transporters that are needed to import essential nutrients. The non-gene associated reactions were assumed to also be present in C. griseus. The following steps were performed in order to determine which of the 3919 gene associated reactions should also be present in the C. griseus model.

Reconstruction of a C. griseus Metabolic Model

Protein homologs in C. griseus for the 2194 genes in Recon 2 were found using a 2-way blast comparison between the human proteome and the C. griseus proteome. The Recon 2 genes are primarily provided as NCBI gene IDs. All but 134 Recon 2 gene IDs that were found to be associated with at least one protein sequence in C. griseus. The remaining 134 were manually analyzed to identify whether the lack of a C. griseus match was due to a missing gene in C. griseus or a problem with the Recon 2 gene ids. 23 appeared to be incorrect gene IDs in Recon 2 that were not directly associated with at least one protein sequence: 1 was a plant gene, 12 were pseudogenes, 3 were mouse genes, 1 was non-coding RNA and 6 had been removed from the NCBI databases for a variety of reasons. The remaining 111 were mainly EST ID numbers which could also be associated with at least one protein sequence by either direct association in the NCBI databases or by blasting the EST sequence against the human genome. Left were a small number of unknown IDs, 3 pseudogenes, 3 genomic contig identifiers. Anything that could not be directly associated with a protein sequence was henceforth treated as a non-gene associated reaction.

A 2 way blast was then performed which identified C. griseus homologs associated with all but 361 reactions in Recon 2. These 361 reactions were manually analyzed. 58 of the reactions were determined to most likely not be present in C. griseus based on a lack the genes present in the C. griseus genome.

In addition, a reaction for cytidine monophospho-N-acetylneuraminic acid hydroxylase (Cmah) was added, since the enzyme is known to occur in C. griseus. This enzyme catalyzes the reaction cmp-NeuAc->cmp-NeuGc through a redox reaction. This C. griseus model was then analyzed and additional modifications were made to the handling of electrons in the mitochondrial electron transport chain and the reaction ARTPLM2 (reaction identifiers are consistent with those reported on the world wide web at humanmetabolism.org unless otherwise noted) was changed to make it reversible.

Tailoring of Cell Line Specific Models Based on Transcriptomics Data

Transcriptomic (RNAseq) data was used to create cell line specific models. RNA-Seq from CHO-K1 and CHO-S was analyzed to determine the presence or absence of transcription of all the genes in the genome. Of the genes used in the C. griseus model, 515 genes in CHO-S and 506 genes in CHO-K1 were determined to not be transcribed. Using GIMME (Becker et al. Plos Comp bio, 2008) with the RNA-Seq data, functioning models were obtained with 4940 reactions for CHO-K1 and 4918 reactions for CHO-S.

Validation of Metabolic Models

The models were validated using external nutrient uptake and secretion data from Selvarasu et al. (Biotechnol Bioeng. 109(6):1415-29 (2012)) to predict growth rate. Experimentally measured doubling time was 40 hours and model predicted cell doubling time was 42 hours.

Model Preparation for Integration with the Glycosylation Network

As the reconstruction has been based on human Recon 2, several reactions required minor corrections to enable the synthesis of glycans. Metabolite abbreviations are defined at bigg.ucsd.edu and humanmetabolism.org.

To allow for the reconsumption of the glucose-mannose complex removed during the initial stage of glycosylation, reactions hydrolyzing the saccharide bonds were added, breaking the glc1man into 1 glucose and 1 mannose, the glc2man into 2 glucoses and one mannose, and the glc3man into 3 glucoses and one mannose. All reactions used a water molecule for each hydrolyzed saccharide bond.

The model could not create ump in the Golgi, which is needed for the uacgam-ump antiporter between the Golgi and cytosol, despite having the reactions to do so. To enable ump synthesis, the reaction NDP7g needs to be enabled. The reaction is identical to the UDPase reported activity in the rat mammary Golgi, so is highly likely also to be active in CHO. The NDP7g reaction has a byproduct of H⁺, and as no other Golgi reactions in the model use H⁺, a proton leak needs to be added allowing the leak of protons from the Golgi to the cytosol, simulating the tight pH control of Golgi transporters.

Furthermore, gdpfuc should be transported from the cytosol to the Golgi with a gmp antiporter, but as the gmp cannot be made in the Golgi a reaction to create gmp must be added. It is possible to create gmp from gdp through hydrolysis in a manner similar to the NDP7g reaction using a nucleoside diphosphatase. By enabling the change from gdp to gmp, the gmp can be returned to the cytosol and more gdpfuc can be transported into the Golgi.

In preparation for the integration of the glycosylation network to the metabolic network, all N-linked glycosylation reactions in the Golgi in the metabolic model were removed (but later re-added in the naming convention from the glycosylation network). A full list of these reactions that were deleted can be found in Table 2.

TABLE 2
List of Reaction Identifiers Used for Deleted Reactions

| | | | |
|---|---|---|---|
| F6Tg | G14Tg | M1316Mg | M13N2Tg |
| M13N4Tg | M14NTg | M16N4Tg | M16N6Tg |
| M16NTg | MM5ag | MM5bg | MM5cg |
| MM6B1ag | MM6B1bg | MM6B2g | MM6ag |
| MM6bg | MM7Ag | MM7B1g | MM7B2g |
| MM7Cag | MM7Cbg | MM8Ag | MM8Cg |

Glycosylation Naming and Standards for the Network

The glycosylation network was created based on The Consortium for Functional Glycomics suggested nomenclature. This nomenclature is a modified version of the condensed IUPAC naming standard for carbohydrates. The linear representation was adopted (world wide web at functionalglycomics.org/static/consortium/Nomenclature.shtml). While the network developed here focuses on N-linked glycosylation, the method utilized could be used to generate a glycosylation network with any other form of glycan naming nomenclature or structural nomenclature that a) provide exactly one unique name or structure for each unique glycan, and that b) is capable of being represented in a single text string, either directly or through a dictionary translating the structure or representation to a string. The nomenclature could be representations in Glycominds Linear Code®, IUPAC or Extended IUPAC, CarbBank™, KCF™, LINUCS™, BCSDB™, InChI™ ClycoCt™ formats, or XML.

For the model to be prepared for appending the network, its current N-linked glycans must be removed (Table 2) since these are added with the entire glycan network. A set of initial glycosylation reactions are kept with the product glycan names being replaced to make it uniform with the chosen naming paradigm of the glycosylation network. A full list of reactions with altered glycan products can be found in Table 3.

TABLE 3
Glycan Reactions with Glycan Product Name based on the
Center for Functional Glycomics Condensed IUPAC Naming Standard

| | Previous | |
|---|---|---|
| Reaction | glycan | |
| identifier | name | Updated glycan name |
| ENMAN1g | m7masnA | Mana1-2Mana1-3(Mana1-2Mana1- |
| | | 3(Mana1-2Mana1-6)Mana1-6)Manb1- |
| | | 4GlcNAcb1-4GlcNAc; Asn |
| ENMAN2g | m7masnA | Mana1-2Mana3(Mana1-2Mana1- |
| | | 3(Mana1-2Mana1-6)Mana1-6)Manb1- |
| | | 4GlcNAcb1-4GlcNAc; Asn |
| ENMAN3g | m7masnA | Mana1-2Mana1-3(Mana1-2Mana1- |
| | | 3(Mana1-2Mana1-6)Mana1-6)Manb1- |
| | | 4GlcNAcb1-4GlcNAc; Asn |
| ENMAN4g | m6masnC | Mana1-2Mana1-3(Mana1-2Mana1- |
| | | 3(Mana1-6)Mana1-6)Manb1- |
| | | 4GlcNAcb1-4GlcNAc; Asn |
| ENMAN5g | m6masnB2 | Mana1-2Mana1-3(Mana1-3(Mana1- |
| | | 2Mana1-6)Mana1-6)Manb1- |
| | | 4GlcNAcb1-4GlcNAc; Asn |
| ENMAN6g | m5masnB1 | Mana1-2Mana1-3(Mana1-3(Mana1- |
| | | 6)Mana1-6)Manb1-4GlcNAcb1- |
| | | 4GlcNAc; Asn |
| M8MASNterg | m8masn | Mana1-2Mana1-2Mana1-3(Mana1- |
| | | 2Mana1-3(Mana1-2Mana1-6)Mana1- |
| | | 6)Manb1-4GlcNAcb1-4GlcNAc; Asn |
| M7MASNBterg | m7masnB | Mana1-2Mana1-2Mana1-3(Mana1- |
| | | 3(Mana1-2Mana1-6)Mana1-6)Manb1- |
| | | 4GlcNAcb1-4GlcNAc; Asn |

Generating a Glycosylation Reaction Network

The glycosylation network was created in two steps, starting with the network generation, and followed by trimming of the network based on experimentally measured glycans. Sugar-residue linkages known to be present in N-linked glycosylation in any species were used to create an initial set of glycosyltransferase-catalyzed reactions, based on enzymatic rules defined by The Consortium for Functional Glycomics (world wide web at functionalglycomics.org/glycomics/molecule/j sp/glycoEnzyme/geMolecule.jsp). This, for example, would link a glycosyltransferase such as GnTI to all reactions it can catalyze. The set of glycosyltransferases and reactions was pruned by removing any pair of glycosyltransferase and reaction where either the glycosyltransferase or its catalyzed linkage was not present in CHO N-linked glycosylation. Furthermore any reaction requiring a precursor that couldn't be created with the remaining reactions was removed. Based on the genomic sequence of the Chinese hamster ovary (CHO)-K1 cell line set forth in Xu et al. (Nature Biotechnology 29, 735-741 (2011)) the only active fucosyltransferase is Fut8 also known as a6FucT. In Hostler et al., (Glycobiology. 19(9):936-49 (2009)), the authors show that the following enzymes are present and active in CHO: N-acetylglucoseamine transferases (GnT) I, II, IV, V; N-acetyllactosaminide β-1,3-N-acetylglucosaminyltransferase (IGnT); both mannosidases (Man) I and II; α3-Sialyltransferase (α3SiaT) is the only expressed sialyltransferase for N-linked glycosylation; and β-1, 4-Galactosyltransferase (b4GalT) is the only present galactosyltransferase. Additional glycosylatransferases are found in the C. griseus genome (Lewis, et al. Nature Biotechnology, 8:759-65 (2013); Xu et al. Nature Biotechnology 29, 735-741 (2011)) and would be used for the synthesis of other glycan structures, including but not limited to O-linked glycans, glycosaminoglycans, GPI anchored glycans, hyaluronan, and glycophingolipids.

The activities of these rules were combined into a set of CHO-specific reaction rules seen in Table 4, and these rules were used to guide the creation of a glycosylation network. The ‘Glycosyltransferase Identifier’ is the enzyme abbreviation that covers a class of the specific enzymes. The ‘Substrate’ is enzyme specificity for a particular glycan. If the substrate is matched within a glycan, the substrate part of the glycan will be replaced by the product. For this sake it is assumed during matching that all antennary branches end with a “(”.

Initially the M9 and M8 structures are the starting glycans to which each substrate in the table is being matched. The resulting glycans are added to a growing list of newly created glycans. This list of glycans will then be the starting point of the next iteration of substrate matching. Table 4 shows these rules based on modified, condensed IUPAC nomenclature, and after each iteration the formed glycans are canonicalized, ensuring that they comply with the naming structure, that substructures with only one branch are not shown as branching, and that no residue has several bonds from the same hydroxy group on any one sugar in the glycan. The method can also be used to generate a glycosylation network with any other naming or structural nomenclature. It can likewise be used to create O-linked glycans, Glycosaminoglycans, Glycosylphosphatidylinositol Anchors, Hyaluronan, and Glycophingolipids.

TABLE 4
List of Genes and the Reaction Rules Associated with the
Genes

| Glycosyl- | | |
|---|---|---|
| transferase | | |
| Identifier | Substrate | Product |
| Fut8 | GlcNAc; Asn | (Fuca6)GlcNAc; Asn |
| GnTI | (Mana1-3(Mana1-3(Mana1- | (GlcNAcb1-2Mana1-3(Mana1- |
| | 6)Mana1-6)Manb1-4 | 3(Mana1-6)Mana1-6)Manb1-4 |
| GnTII | (GlcNAcb1-2Mana1- | (GlcNAcb1-2Mana1- |
| | 3(Mana1-6)Manb1-4 | 3(GlcNAcb1-2Mana1- |
| | | 6)Manb1-4 |
| GnTIV | (GlcNAcb1-2Mana1-3 | (GlcNAcb1-2(GlcNAcb1- |
| | | 4)Mana1-3 |
| GnTV | (GlcNAcb1-2Mana1-6 | (GlcNAcb1-2(GlcNAcb1- |
| | | 6)Mana1-6 |
| ManI | (Mana1-2Mana | (Mana |
| ManII | (Mana1-6Mana | (Mana |
| ManII | (Mana1-3(Mana1-6)Mana | (Mana1-6Mana |
| a3SiaT | Gal | (NeuAca2-3)Gal |
| b4GalT | (GlcNAc | (Galb1-4GlcNAc |
| IGnT | Gal | (GlcNAc1-b3)Gal |

After the network was created, it was trimmed to match experimental data. A list of glycans identified on recombinant biotherapeutics were compiled from literature. These were used to specify the final vertices/nodes in the glycan network (each directed edge in the network is a reaction, and each node is a glycan). By moving against the directionality of the reactions/edges, all paths that synthesize the measured glycans could be identified. All reactions not in these paths were removed from the network to trim it. The resulting glycan network thus contains the identified glycans in CHO, all possible ways for CHO to synthesize these glycans, and all intermediate glycans necessary for arriving at the measured glycans. A full list of the glycans used for trimming can be seen in table 5.

TABLE 5
Full List of Glycans Used for Trimming

| Glycan | |
|---|---|
| ID in | |
| this | |
| patent | Measured glycan representation |
| 1 | GlcNAc?Man?(GlcNAc?Man?)Man?GlcNAc?GlcNAc; Asn |
| 2 | Man?(GlcNAc?Man?)Man?GlcNAc?(Fuc?)GlcNAc; Asn |
| 3 | Man?(Gal?GlcNAc?Man?)Man?GlcNAc?(Fuc?)GlcNAc; Asn |
| 4 | GlcNAc?Man?(GlcNAc?Man?)Man?GlcNAc?(Fuc?)GlcNAc; Asn |
| 5 | GlcNAc?Man?(Gal?GlcNAc?Man?)Man?GlcNAc?(Fuc?)GlcNAc; Asn |
| 6 | Gal?GlcNAc?Man?(NeuAc?Gal?GlcNAc?Man?)Man?GlcNAc?GlcNAc; Asn |
| 7 | Gal?GlcNAc?Man?(Gal?GlcNAc?Man?)Man?GlcNAc?(Fuc?)GlcNAc; Asn |
| 8 | NeuAc?Gal?GlcNAc?Man?(NeuAc?Gal?GlcNAc?Man?)Man?GlcNAc?GlcNAc; Asn |
| 9 | NeuAc?Gal?GlcNAc?Man?(Gal?GlcNAc?Man?)Man?GlcNAc?(Fuc?)GlcNAc; Asn |
| 10 | NeuAc?Gal?GlcNAc?Man?(NeuAc?Gal?GlcNAc?Man?)Man?GlcNAc?(Fuc?)Glc |
| | NAc; Asn |
| 11 | Gal?GlcNAc?Man?(Gal?GlcNAc?(Gal?GlcNAc?)Man?)Man?GlcNAc?(Fuc?)GlcNAc; |
| | Asn |
| 12 | Gal?GlcNAc?Man?(NeuAc?Gal?GlcNAc?(Gal?GlcNAc?)Man?)Man?GlcNAc?(Fuc?) |
| | GlcNAc; Asn |
| 13 | Gal?GlcNAc?Man?(Gal?GlcNAc?Gal?GlcNAc?(Gal?GlcNAc?)Man?)Man?GlcNAc? |
| | (Fuc?)GlcNAc; Asn |
| 14 | NeuAc?Gal?GlcNAc?Man?(NeuAc?Gal?GlcNAc?(Gal?GlcNAc?)Man?)Man?GlcNAc? |
| | (Fuc?)GlcNAc; Asn |
| 15 | NeuAc?Gal?GlcNAc?Man?(NeuAc?Gal?GlcNAc?(NeuAc?Gal?GlcNAc?)Man?)Man? |
| | GlcNAc?(Fuc?)GlcNAc; Asn |
| 16 | NeuAc?Gal?GlcNAc?(Gal?GlcNAc?)Man?(NeuAc?Gal?GlcNAc?(NeuAc?Gal?Glc |
| | NAc?)Man?)Man?GlcNAc?(Fuc?)GlcNAc; Asn |
| 17 | NeuAc?Gal?GlcNAc?(NeuAc?Gal?GlcNAc?)Man?(NeuAc?Gal?GlcNAc?(NeuAc? |
| | Gal?GlcNAc?)Man?)Man?GlcNAc?(Fuc?)GlcNAc; Asn |
| 18 | NeuAc?Gal?GlcNAc?(Gal?GlcNAc?Gal?GlcNAc?)Man?(NeuAc?Gal?GlcNAc?(Neu |
| | Ac?Gal?GlcNAc?)Man?)Man?GlcNAc?(Fuc?)GlcNAc; Asn |
| 19 | NeuAc?Gal?GlcNAc?(NeuAc?Gal?GlcNAc?Gal?GlcNAc?)Man?(NeuAc?Gal?Glc |
| | NAc?(NeuAc?Gal?GlcNAc?)Man?)Man?GlcNAc?(Fuc?)GlcNAc; Asn |
| 20 | NeuAc?Gal?GlcNAc?(NeuAc?Gal?GlcNAc?Gal?GlcNAc?)Man?(NeuAc?Gal?Glc |
| | NAc?Gal?GlcNAc?(NeuAc?Gal?GlcNAc?)Man?)Man?GlcNAc?(Fuc?)GlcNAc; Asn |

The glycans have been found in EPO and/or IgG produced in CHO strains. ‘?’ shows an undetermined linkage from the original data source (i.e., when the link between two sugars is known but the stereochemistry and/or hydroxyl group in the link is unclear). These were replaced by glycans with the given structure that could be built with the enzyme rules from M9. Since the linkages are undefined, al-6 and al-3 branches from mannose can be swapped in their order in a string. Thus, the glycans are not unique representations.

### Example 3

### Optimized Recombinant Protein Titers

The model of Example 2 can be used to identify optimal growth conditions (e.g., optimal growth rate for production) to ensure the highest theoretical conversion of a carbon source to a biological molecule of interest.

By setting the model to grow at specific growth rates, the theoretical optimal IgG production rate can be determined. Combining this with the growth rate (or doubling time), a chosen length of fermentation, and exponential growth function, the theoretical maximal IgG conversion from a carbon source can be found.

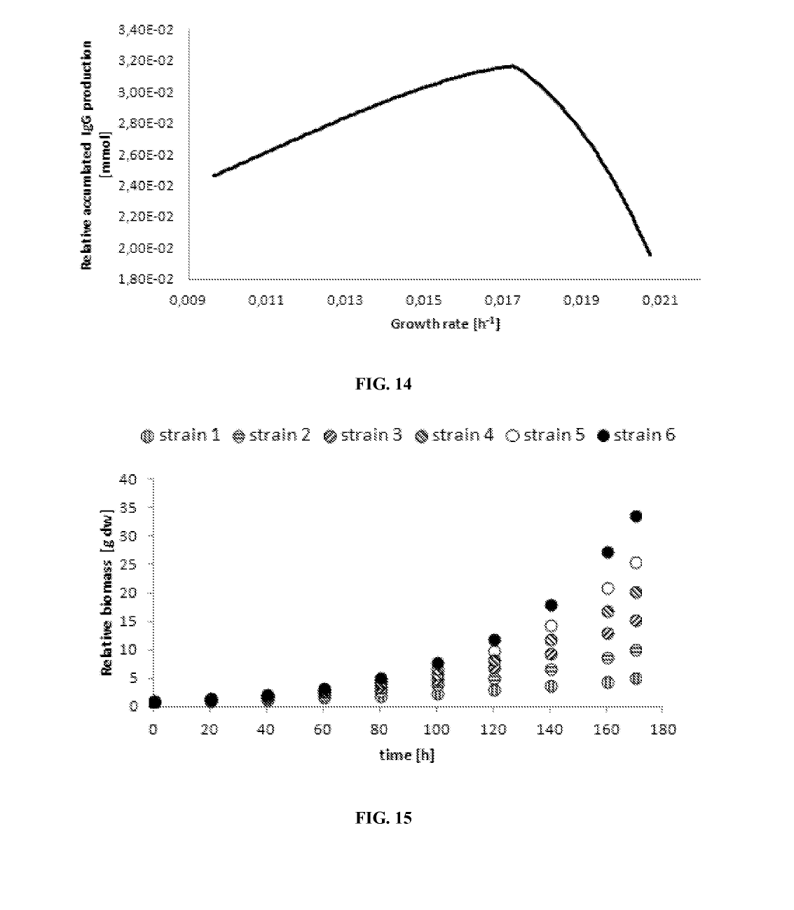

Over a 168 h fermentation the growth rate of 0.01724 with a theoretical maximal IgG production of 3.192·10⁻⁵ mmol/g dw/h can produce much more IgG, than when CHO is growing at its maximal predicted growth rate (FIG. 14).

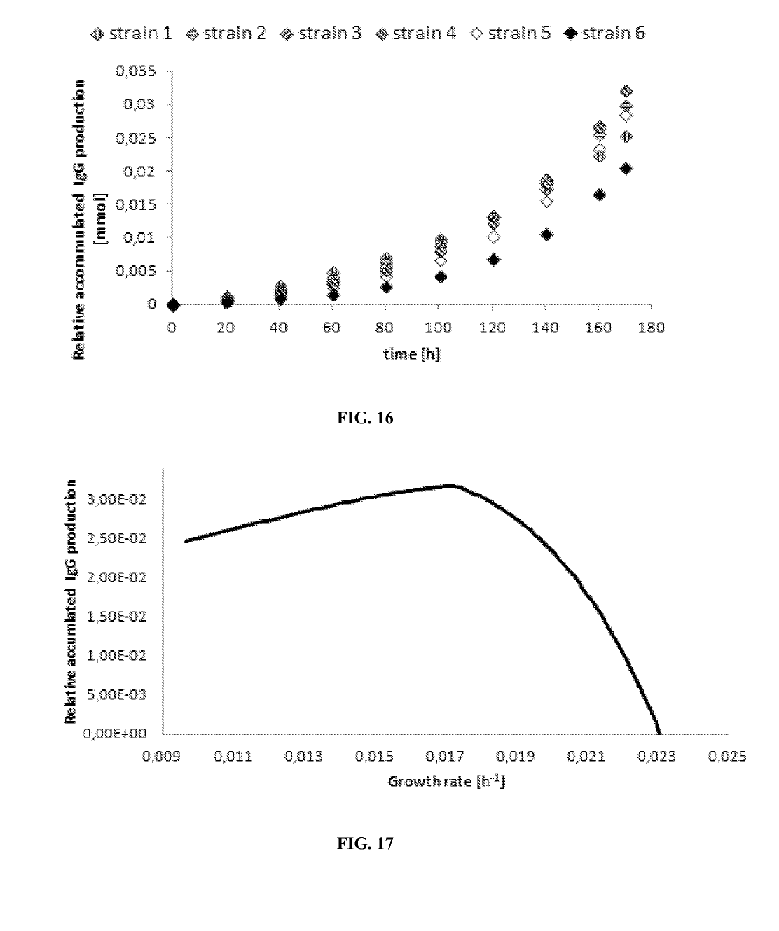

Alternatively the model can be used to identify the optimal strain for production of a biological molecule of interest such as IgG. By simulating clones with different growth rates producing IgG, and using the integral of the growth function, the accumulated theoretical maximal conversion of carbon sources to IgG can be derived. FIG. 15 shows the relative biomass production of the 6 different clones (or strains), indexed to an inoculated culture mass of 1 gram dry weight (g dw), and FIG. 16 shows the IgG yields corresponding to these clones (or strains).

Optimize Protein Titers in the CHO-K1 Specific Model

The model can also be used to identify the optimal growth conditions or growth rate that ensures the highest theoretical conversion of a carbon source to a biological molecule of interest. These molecules could be amino acids, nucleotides, lipids, recombinant proteins, etc. Here it is demonstrated for IgGs.

By setting the model to grow at specific growth rates it can be used to solve for the theoretical optimal IgG production flux. Combining this with the growth rate, a chosen length of fermentation, and the growth function, the theoretical maximal IgG conversion from a carbon source can be found.

Over a 168 h fermentation the growth rate of 0.01724 with a theoretical maximal IgG production of 3.19·10⁻⁵ mmol/g dw/h can produce a maximal amount of IgG (FIG. 17).

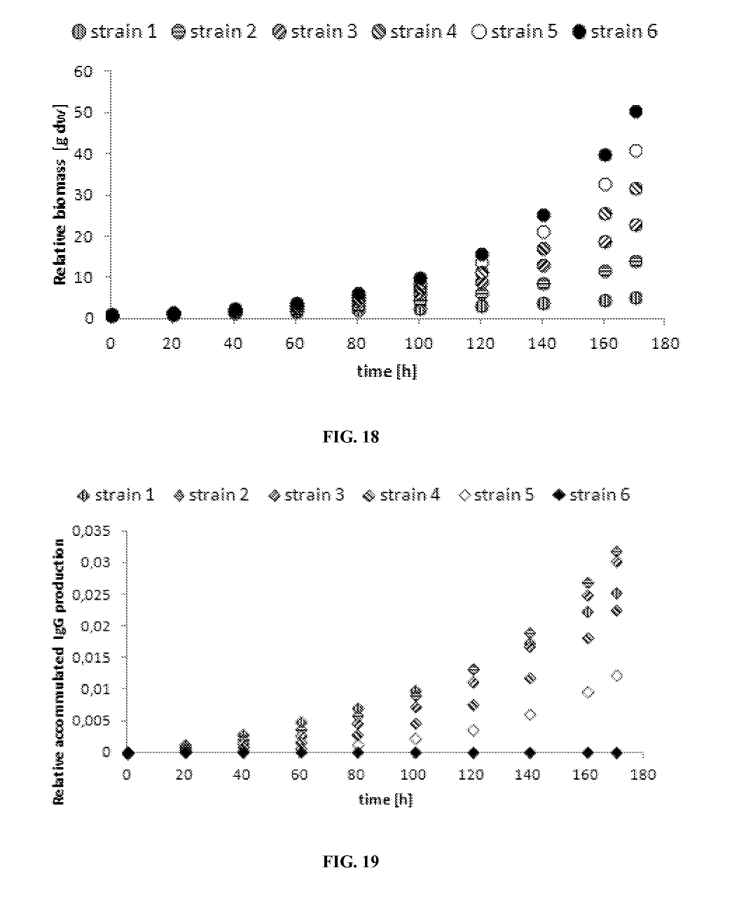

Alternatively the model can be used to identify the optimal strain for production of a biological molecule of interest such as IgG. By simulating the strains with different growth rates producing IgG, and using the integral of the growth function, the accumulated theoretical maximal conversion of carbon sources to IgG can be derived. FIG. 18 shows the relative biomass production of the 6 different strains, indexed to an inoculated culture mass of 1 g dw, and FIG. 19 shows the amount of IgG produced.

Optimize Protein Titers in the CHO-S Specific Model

A similar analysis was conducted on the model that was tailored to CHO-S metabolism based on its gene expression. By setting the CHO-S model to grow at specific growth rates it can be used to solve for the theoretical optimal IgG production yield. Combining this with the growth rate, a chosen length of fermentation, and the growth function, the theoretical maximal IgG conversion from a carbon source can be found.

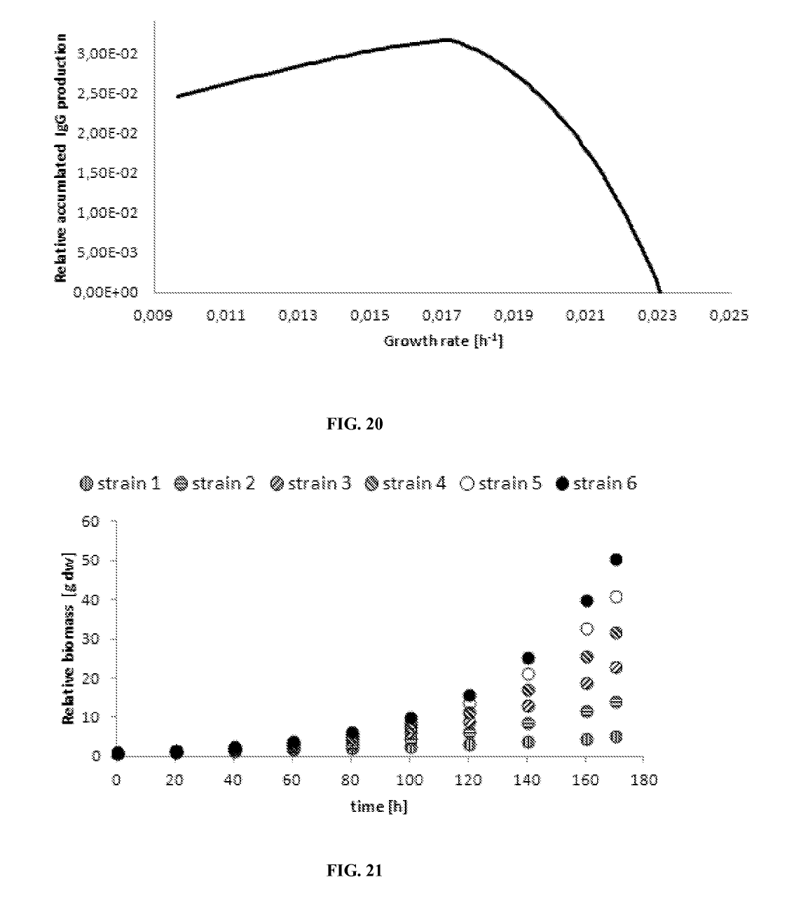

Over a 168 h fermentation the growth rate of 0.01724 with a theoretical maximal IgG production of 3.192·10⁻⁵ mmol/g dw/h can produce much more IgG, than the fastest growing CHO (FIG. 20).

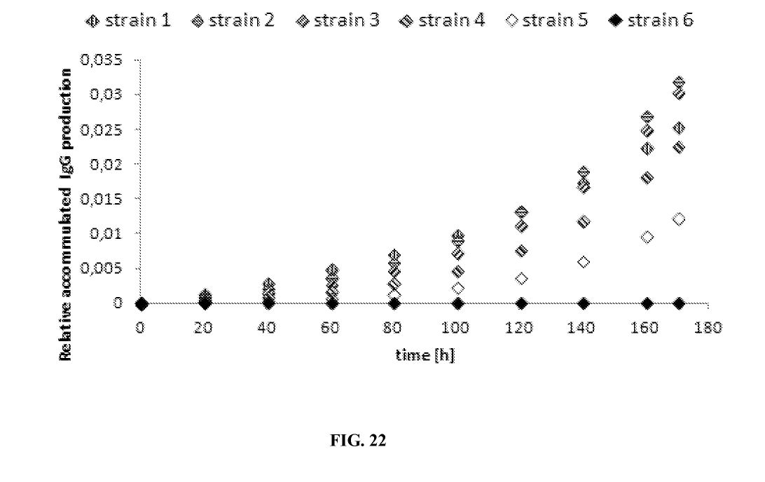

Alternatively the model can be used to identify the optimal clones or strains for production of a biological molecule of interest such as IgG. By simulating the clones or strains with different growth rates producing IgG, and using the integral of the growth function, the accumulated theoretical maximal conversion of carbon sources to IgG can be derived. FIG. 21 shows the relative biomass production of the 6 different strains, indexed to an inoculated culture mass of 1 g dw, and FIG. 22 shows the amount of IgG produced in CHO-S.

### Example 4

### Simulating the Coupled Metabolic/Glycosylation Model

All reactions in the coupled metabolic/glycosylation network were tested for the ability to carry flux using a max/min optimization problem for each reaction in the network as in Burgard et al. (Biotechnol Prog. 17(5):791-7 (2001)). For this, 58% of the metabolic reactions were functional, and 100% of all glycosylation reactions were active.

Prediction on how Glycosyltransferase Knockouts Change Glycoform

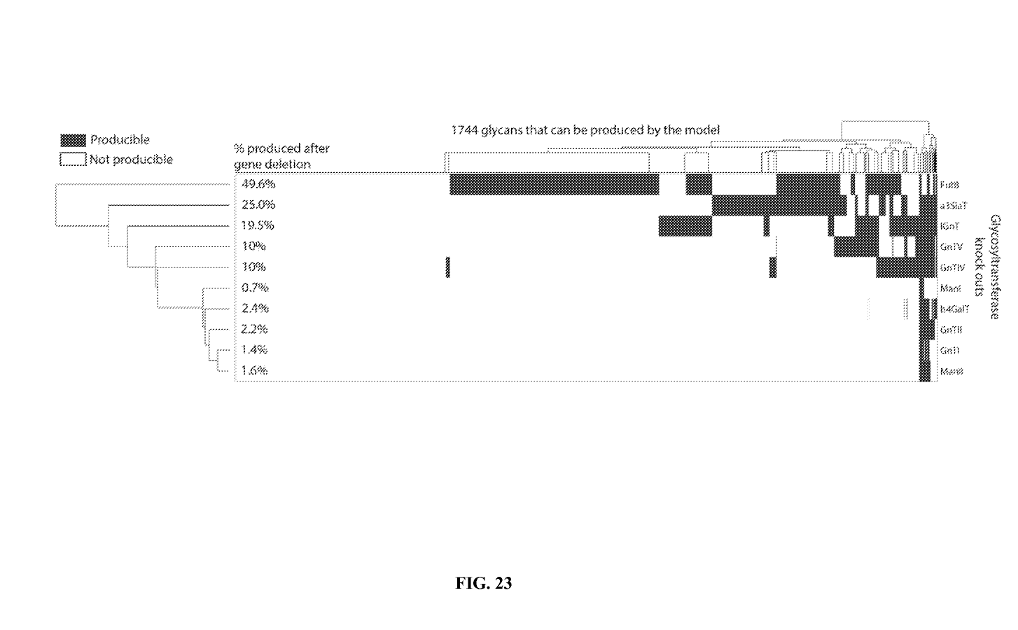

More than 1000 genes are known to influence glycosylation either through the synthesis of sugar nucleotide subunits, transport of molecules across membranes, the polymerization of subunits into glycans, or degradation of glycans. A series of glycosyltransferase enzymes are directly responsible for polymerization. However, because of the combinatorial activity of the glycosyltransferases, it is not obvious how the deletion of each glycosyltransferase impacts which glycans cannot be synthesized when each enzyme is removed. Thus, with the coupled metabolic/glycosylation network, each class of glycosyltransferases responsible for N-linked glycosylation was removed and the biosynthetic capabilities for all 1744 glycans in the model were tested. Each glycosyltransferase knockout yielded a different profile of glycans that could be produced, and greatly decreased the number of producible glycans (FIG. 23).

Prediction on how Metabolic Knockouts Change Glycoform

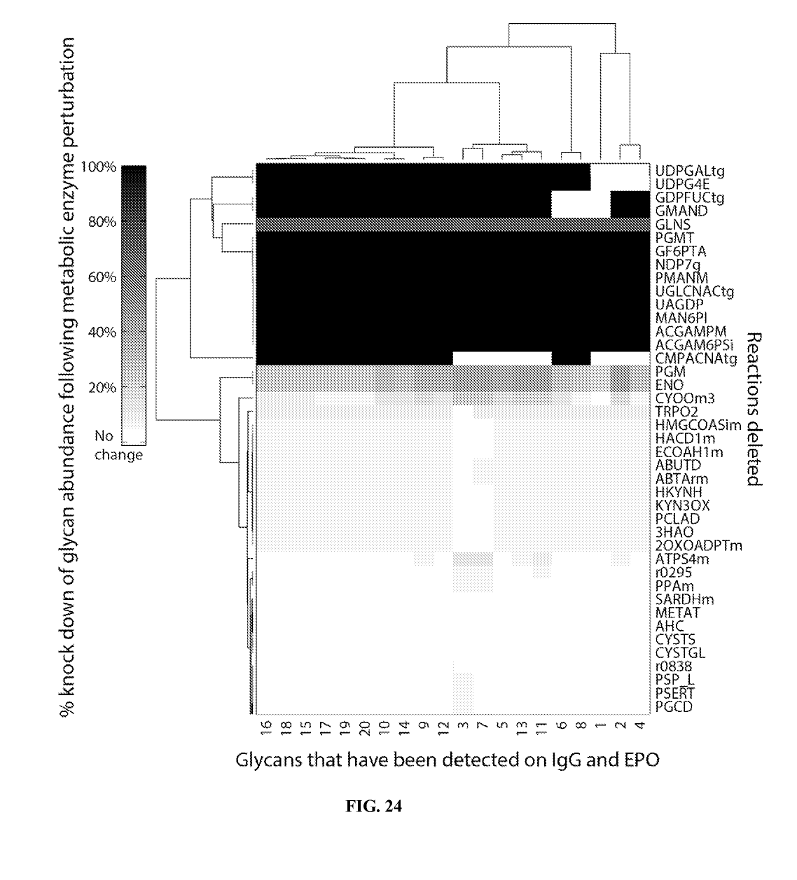

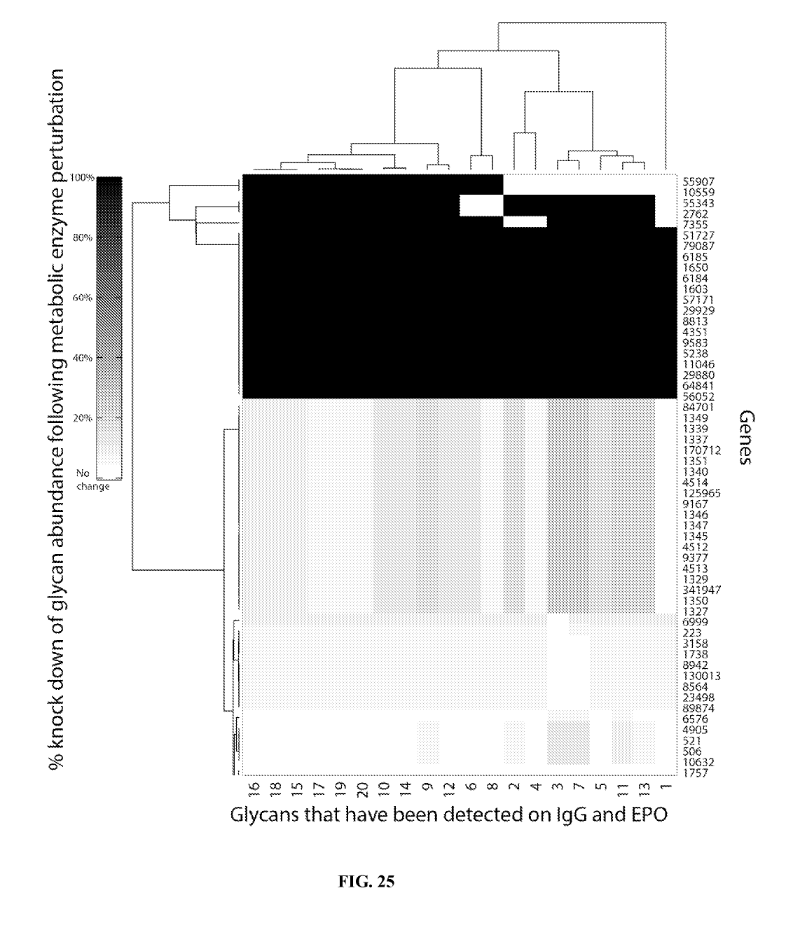

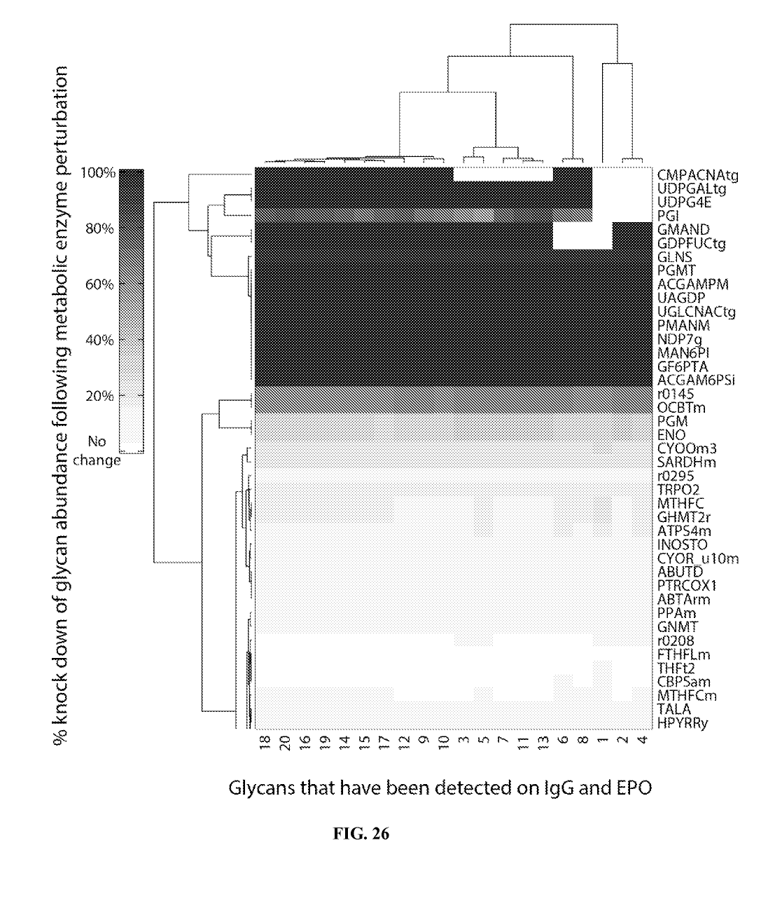

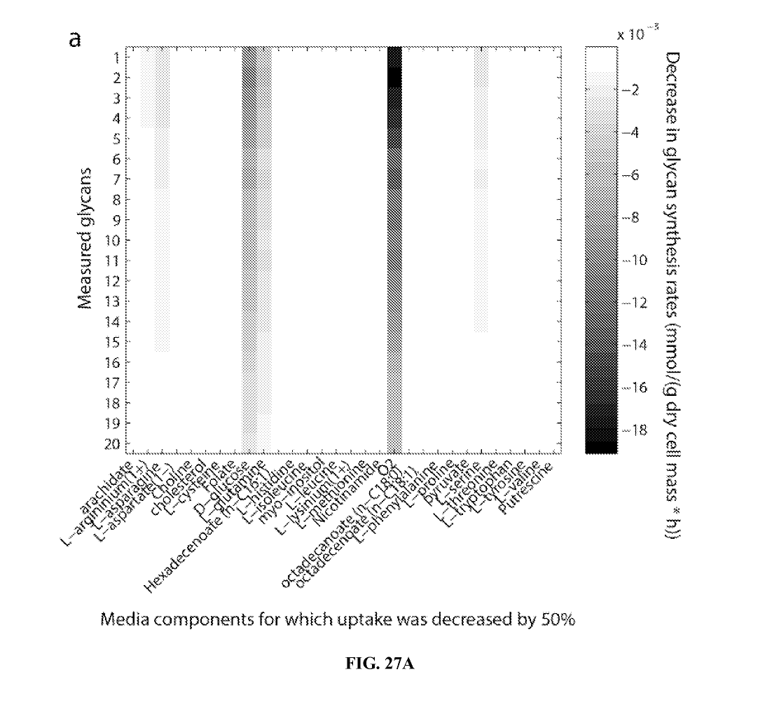

Glycosylation requires the coordinated activity of metabolic enzymes and transporters that synthesize and transport glycan precursors. Given the complexity of metabolism, it is not immediately clear how such enzymes and transporters might influence the synthesis of specific glycans. Thus the integrated glycosylation/metabolic model was used to identify enzymes and transporters that would influence the synthesis of different glycans in non-obvious ways. This could be done by systematically removing each reaction or gene in the network, and then simulating the amount of each glycan that can be made. To do this the model was first set to the optimal biomass objective function value to mimic exponential growth. Second, an enzymatic reaction was knocked out, and then the maximum production yield was computed for each glycan that was measured on IgG and EPO that were recombinantly synthesized in CHO cells. This was repeated for each enzyme-catalyzed (or transporter-facilitated) reaction in the model. From this simulation, a list of non-obvious enzyme deletions that removed the ability to synthesize specific glycans and altered the amount of other glycans that could be produced was identified (FIG. 24). This analysis was repeated for gene knockouts (FIG. 25), and also for reaction knocks after varying media inputs (FIG. 26).

For data set forth in FIG. 24, enzymatic reactions and transporters were removed from the model, and the ability to synthesize 20 experimentally measured glycans was tested. The maximal synthesis for each glycan was normalized to the wild-type synthesis rates. Only the metabolic perturbations that had the greatest effect on glycosylation are shown, and reaction identifiers are detailed in the Recon 2 model from Thiele et al. (Nature Biotechnology 31(5):419-25 (2013)). Some reactions/transporter perturbations removed specific glycans. For example, the Golgi transporter for cmp-NeuAc (CMPACNAtg) blocked the synthesis of sialylated glycans, and the removal of the gdp-fucose transporter (GDPFUCtg) removed fucosylated glycans. However, many non-obvious enzyme deletions (e.g., PGM—phosphoglycerate mutase, ENO—enolase, and CYOOm3—cytochrome-c oxidase, GMAND—GDP-D-mannose dehydratase, UDPG4E—UDPglucose 4-epimerase) blocked other specific glycans or reduced the efficiency of making certain glycans more than others.

For data set forth in FIG. 25, each gene was removed from the model, and the ability to synthesize 20 experimentally measured glycans was tested. The maximal synthesis for each glycan was normalized to the wild-type synthesis rates. Here just the genetic perturbations that had the greatest effect on glycosylation are shown, and genes correspond to Entrez Gene™ identifiers for human homologues, which are associated to the hamster genes through sequence homology or comparison of gene annotation. Some gene deletions removed specific glycans. For example, the Golgi transporter for cmp-NeuAc (10559) blocked the synthesis of sialylated glycans, and the removal of the gdp-fucose transporter (55343) removed fucosylated glycans. However, many non-obvious enzyme deletions (e.g., cytochrome-c oxidase, GDP-D-mannose dehydratase, etc.) blocked other specific glycans or reduced the efficiency of making certain glycans more than others.

For data set forth in FIG. 26 enzymatic reactions and transporters were removed from the model, and the constraints on media uptake were relaxed for 12 components that were not measured. Then the reaction deletion analysis was repeated. When testing the ability to synthesize 20 experimentally measured glycans, the combinations of media and enzyme perturbations influenced glycan synthesis in sometimes non-obvious ways (see for example, PGI—glucose-6-phosphate isomerase, OCBTm—ornithine carbamoyltransferase, PGM—phosphoglycerate mutase, ENO—enolase, GMAND—GDP-D-mannose dehydratase, UDPG4E—UDPglucose 4-epimerase). Some of these combinations blocked specific glycans or reduced the efficiency of making certain glycans more than others. Reaction identifiers are detailed in the Recon 2 model from Thiele et al. (Nature Biotechnology 31(5):419-25 (2013)).

Prediction on how Variations in Media Components Change Glycoform

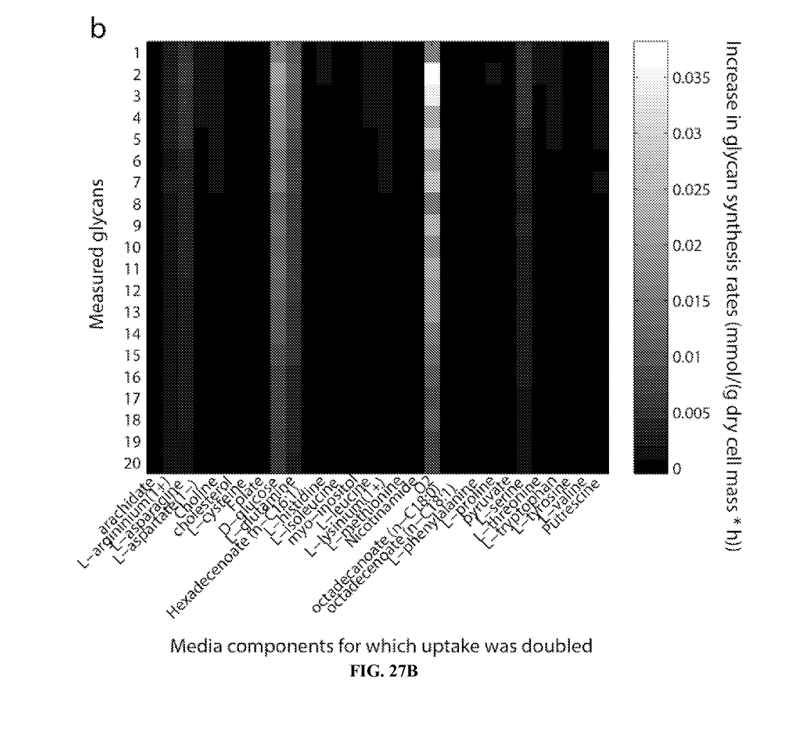

Media provides all metabolic precursors necessary for growth and the synthesis of all cell parts, and indirectly influences the energy balance, which further influences which metabolic pathways are used by the cell. Furthermore, studies have demonstrated that one can influence the cell growth and the glycoprofile of a recombinant protein by altering media conditions. Thus, it is likely that the metabolic/glycosylation model can be used to predict media changes that will influence the synthesis of glycans. To test this, the uptake rates of metabolites that were measured during growth of a CHO cell line producing recombinant protein were determined, and then flux balance analysis was used to predict missing uptake rates for unmeasured metabolites that were required for growth and/or in the media. Then these uptake rates for each metabolite were used to simulate the synthesis of the 20 measured glycans. This analysis was repeated after decreasing the amount of uptake for each individual metabolite to 50% of the uptake in the base medium, and also after increasing the uptake by 2×. Since increases and decreases in the uptake of individual components can increase or decrease the amount of carbon or energy for all glycans, the magnitude of change for all glycans was assessed by subtracting off the synthesis rate for the base medium. This allowed for the comparison of all glycans side by side to identify glycans that changed more or less than all other glycans. Several compounds such as amino acids, inositol, and aeration levels increased or decreased the rate of synthesis of individual glycans (more so than other glycans), as shown in FIG. 27. This demonstrated that non-intuitive connections between individual glycans and individual media components can be found, and that media might be formulated to enrich for desired glycans.

For data set forth in FIG. 27, individual media components were doubled or decreased to half of their measured or predicted based medium uptake rates and the synthesis rates of each of 20 measured glycans were predicted for the new media formulations. The synthesis rates were then compared to the synthesis rates of the base medium to identify which media components would bias the synthesis of specific glycans. Several glycans were enriched compared to others under some changes in media.

### Example 5

### Analysis of Cell-Line Specific Metabolic Models

Using GIMME (Becker et al. PLoS Comput Biol. 4(5):e1000082 (2008)), the CHO-K1 and CHO-S specific models were built based on RNA-Seq data acquired from the two different cell lines. These models were subjected to all of the same analyses described above, including the assessment of glycan synthesis capacities following gene deletions or variations in media. In these analyses, the cell-line specific models showed some cell-line specific behavior. For example, when screening gene deletion knockouts, the knockout of glucosamine (UDP-N-acetyl)-2-epimerase/N-acetylmannosamine kinase (GNE) decreases the ability for CHO-S to make sialylated glycans, while its knockout shows no effect on sialylation in the CHO-K1 model.

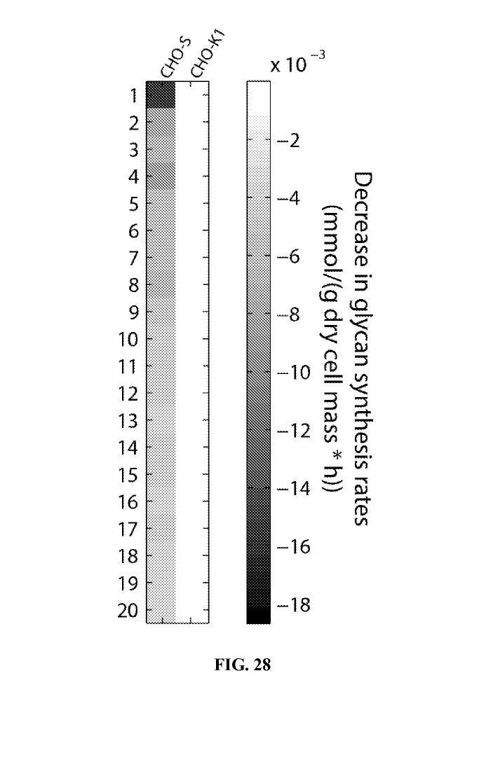

When screening variations in media components, CHO-S glycosylation shows an increased sensitivity to reductions in myo-inositol availability. Reductions in its uptake significantly affect glycosylation in CHO-S cell lines, while there is little effect on CHO-K1 glycosylation when subjected to large variations in myo-inositol uptake (FIG. 28). Decreasing the uptake of myo-inositol to 50% of the uptake rate observed in the models leads to significant decreases in glycan synthesis capacities in CHO-S but not in CHO-K1 (FIG. 28). This most strongly impacted the synthesis of GlcNAc?Man?(GlcNAc?Man?)Man?GlcNAc?GlcNAc;Asn in CHO-S cells.

### Example 6

### Analysis of Cell-Line Specific Mutations

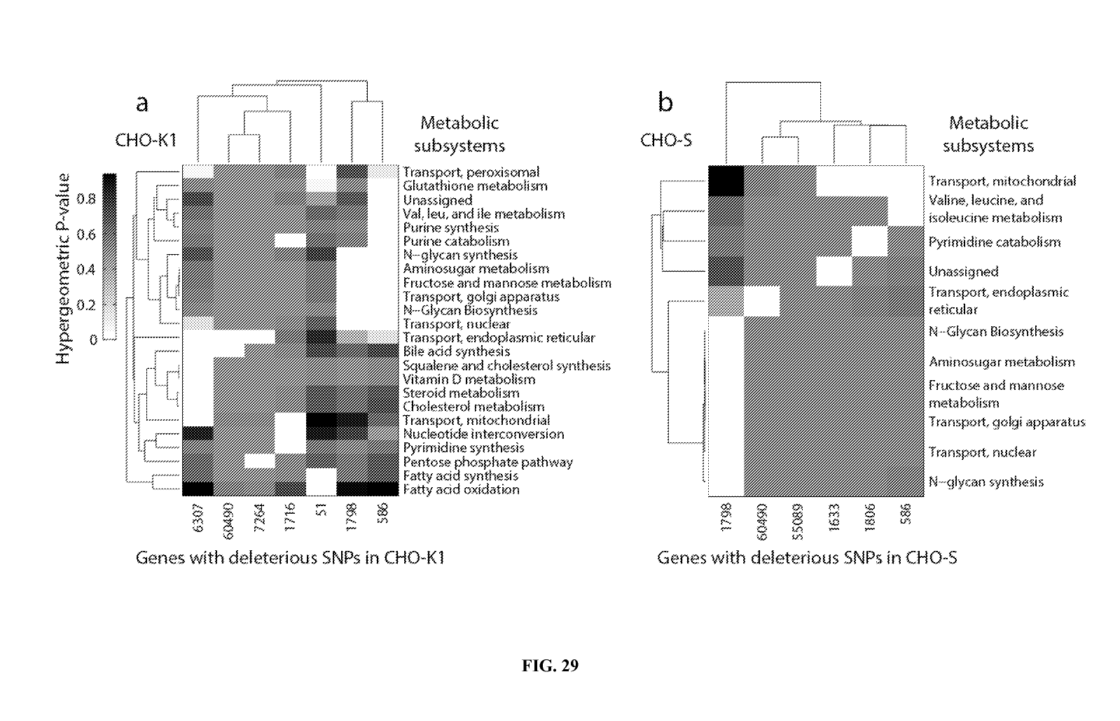

Each cell line has millions of mutations when compared to the parent hamster genome. Many of these are found in metabolism, and a subset is unique to specific cell lines. For example, the dihyrofolate reductase (DHFR) is deleted in DG44 cell lines and used as a metabolic enzyme-based selection system to induce higher titers of recombinant protein expression. Among all mutations, roughly % are shared among all cell lines, and the remaining ⅓ vary among the cell lines. Mutations can negatively affect enzyme activities, which might lead to differences in metabolic capabilities among host cell lines. Here, lists of nonsynonymous SNPs in the ATCC CHO-K1 and CHO-S cell lines were compiled. For the two cell lines, 103 metabolic genes in C. griseus had non-synonymous SNPs (94 in CHO-K1 and 84 in CHO-S). Deleterious SNPs were identified using Provean (Choi et al. PLoS One. 7(10):e46688 (2012)), and the algorithm predicted 15 CHO-K1 and 13 CHO-S genes to be disrupted. After incorporating these SNPs into the RNA-Seq tailored CHO-K1 and CHO-S models described in Example 5, and after accounting for isozymes, 7 genes with deleterious SNPs in CHO-K1 would block metabolic reactions, and 6 mutated genes in CHO-S would block metabolic reactions in the respective models. The systemic effects of deleterious mutations analyzed using the cell-line specific models by removing the genes with mutations from the CHO-K1 and CHO-S models. When each deleterious mutation was accounted for in the models, they do not affect predicted growth. While these endogenous mutations do not directly affect growth, they do affect the utilization of different metabolic pathways in the CHO-K1 and CHO-S genomes. Focusing on mutations in metabolism, the effects of each mutation on different metabolic pathways in CHO cells were tested, thus providing detailed insights into how the mutations unique to each cell line likely influences cell-line specific metabolic pathway usage. To do this, we knocked out each mutated gene from the cell line specific models and used flux variability analysis to assess how each knockout affected all other pathways. In doing this, all reactions that had at least a 5% decrease in their flux span (i.e., the difference between the maximum and minimum predicted metabolic flux levels for a given reaction) were identified and then a hypergeometric test was used to identify metabolic subsystems with more affected reactions than expected by random chance (p<0.01). These are all reported for the mutations in CHO-K1, simulated in the CHO-K1 model (FIG. 29.a), and CHO-S mutations in the CHO-S model (FIG. 29.b). For example in CHO-K1 a mutation in deoxyguanosine kinase showed a significant effect on pyrimidine synthesis and purine metabolism. Similarly, a mutation in dihydropyrimidine dehydrogenase in CHO-S has a significant effect on pyrimidine catabolism.

In the model analysis, some mutations affected glycosylation, and so further analysis was done including glycosylation enzymes. For example, our Provean analysis predicted that a mutation in the alpha-2,6-sialyltranferase was deleterious to its function. This result correlates with lack of alpha-2,6 sialylation on glycans produced in CHO-K1 cells. Transfection with an alpha-2,6-sialytransferase is known to correct this deficiency. While each mutation was not predicted to affect growth, this is not unexpected since CHO cells are under growth selection, and so mutations that limit growth would be selected against. However, we demonstrated that the mutations do influence many metabolic pathways, including glycosylation.

Lastly, if however, mutations are found in a cell line with a different phenotype, the impact of the mutation can be assessed in the same manner. That is, the simulation of a phenotype can be conducted with and without the reaction catalyzed by the mutated enzyme, to assess the impact of the mutation on a phenotype of interest. For example, a mutant CHO-K1 cell line (pgsA-745) was identified with a phenotype of not being able to produce heparan sulfate. A SNP in a xylosyltransferase in a CHO-K1 derived cell line (Esko et al. Proc Natl Acad Sci USA. 82: 3197-3201) which reduced xylosyltransferase activity was identified. Removal of this enzyme in the model would lead to a loss of heparin sulfate glycosylation in the CHO model.

Although the invention has been described with reference to the above examples, it will be understood that modifications and variations are encompassed within the spirit and scope of the invention. Accordingly, the invention is limited only by the following claims.

What is claimed is:

1. A method for identifying a Chinese Hamster Ovary (CHO) cell line having a desired genetic trait comprising:
a) providing a sample CHO cell line genome, or portion thereof;
b) comparing the sample CHO cell line genome, or portion thereof, with that of a reference hamster genome to identify at least one of single-nucleotide polymorphisms (SNPs), inversions, indels, and copy number variations (CNVs) in the sample CHO cell line genome, or portion thereof, thereby identifying variations in the sample CHO cell line genome, or portion thereof, associated with the desired genetic trait, wherein the desired genetic trait is related to an improved function relevant to bioprocessing, thereby identifying the CHO cell line as having the desired genetic trait.

2. The method of claim 1, wherein comparing comprises performing computational analysis using a computer generated algorithm.

3. The method of claim 2, wherein the computer generated algorithm is operable to align and map sequence data of the sample CHO cell line genome to that of the reference hamster genome.

4. The method of claim 1, further comprising detecting presence or absence of a desired gene in the sample CHO cell line genome.

5. The method of claim 3, wherein the aligned sequence data is fragmented and sorted according to a mapped position.

6. The method of claim 1, wherein the desired genetic trait is related to cell growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

7. The method of claim 6, wherein the function of the desired genetic trait of the CHO cell line is associated with SNP analysis, inversion analysis, indel analysis, or CNV analysis.

8. The method of claim 6, wherein the desired genetic trait is glycosylation or metabolism.

9. The method of claim 1, wherein the reference hamster genome comprises a nucleic acid sequence as set forth in GenBank Accession Nos: AMDS01000001-AMDS01218862, or portion thereof.

10. A method for generating a desired CHO cell line having a genetic basis for a desired phenotype comprising:
a) providing a sample CHO cell line genome, or portion thereof;
b) comparing the sample CHO cell line genome, or portion thereof, with that of a reference hamster genome to identify at least one of single-nucleotide polymorphisms (SNPs), inversions, indels, and copy number variations (CNVs) in the sample CHO cell line genome, or portion thereof, thereby identifying variations in the sample CHO cell line genome, or portion thereof, associated with a desired phenotype; and
c) introducing one or more genetic changes into the sample CHO cell line to produce the desired CHO cell line having a genetic basis for the desired phenotype, thereby generating the desired CHO cell line having the genetic basis for the desired phenotype.

11. The method of claim 10, wherein comparing comprises performing computational analysis using a computer generated algorithm.

12. The method of claim 11, wherein the computer generated algorithm is operable to align and map sequence data of the sample CHO cell line genome to that of the reference hamster genome.

13. The method of claim 10, further comprising detecting presence or absence of a desired gene in the sample CHO cell line genome.

14. The method of claim 12, wherein the aligned sequence data is fragmented and sorted according to a mapped position.

15. The method of claim 10, wherein the desired phenotype is related to an improved function relevant to bioprocessing.

16. The method of claim 15, wherein the desired phenotype is related to cell growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

17. The method of claim 16, wherein the function of the desired phenotype is associated with SNP analysis, inversion analysis, indel analysis, or CNV analysis.

18. The method of claim 16, wherein the desired genetic trait is glycosylation or metabolism.

19. The method of claim 10, wherein the reference hamster genome comprises a nucleic acid sequence as set forth in GenBank Accession Nos: AMDS01000001-AMDS01218862, or portion thereof.

20. A method for predicting a CHO cell physiological function comprising:
a) providing a data structure associated with a CHO cell physiological function, the data structure relating a plurality of CHO cell reactants to a plurality of CHO cell reactions, wherein each of the CHO cell reactions comprises one or more reactants identified as a substrate of the reaction, one or more reactants identified as a product of the reaction and a stoichiometric coefficient relating the substrate and the product;
b) providing a constraint set for the plurality of CHO cell reactions;
c) providing an objective function; and
d) determining at least one flux distribution that minimizes or maximizes the objective function when the constraint set is applied to the data structure, thereby predicting a CHO cell physiological function related to the gene.

21. The method of claim 20, wherein if at least one flux distribution is not predictive of the CHO cell physiological function, then adding a reaction to or deleting a reaction from the data structure and repeating step (d).

22. The method of claim 20, wherein if at least one flux distribution is predictive of the CHO cell physiological function, then storing the data structure in a computer readable medium.

23. The method of claim 20, further comprising generating a computation model.

24. The method of claim 20, wherein the CHO cell physiological function is selected from the group consisting of growth, biological product production, production of a protein, production of an amino acid, production of a purine, production of a pyrimidine, production of an oligonucleotide, production of a glycan, production of a lipid, production of a fatty acid, production of a bioactive small molecule, transport of a metabolite, and glycosylation of a protein or lipid or fatty acid.

25. The method of claim 20, wherein the data structure comprises a set of linear algebraic equations.

26. The method of claim 20, wherein the flux distribution is determined by linear programming.

27. The method of claim 20, wherein the CHO cell reactions are obtained from a database that includes the substrates, products, and stoichiometry of a plurality of CHO cell reactions.
